(19) United States
(12) Patent Application Publication
(10) Pub. No.: US 2020/0085407 A1
(43) Pub. Date: Mar. 19, 2020

(54) SYSTEMS AND METHODS FOR HARMONIC ACOUSTOGRAPHY FOR QUANTITATIVE MARGIN DETECTION

(71) Applicant: THE REGENTS OF THE UNIVERSITY OF CALIFORNIA, Oakland, CA (US)

(72) Inventors: Warren Grundfest, Los Angeles, CA (US); Maie St. John, Los Angeles, CA (US); Ashkan Maccabi, Tarzana, CA (US); George Saddik, Oxnard, CA (US); Zachary Taylor, Poway, CA (US)

(73) Assignee: THE REGENTS OF THE UNIVERSITY OF CALIFORNIA, Oakland, CA (US)

(21) Appl. No.: 16/582,084

(22) Filed: Sep. 25, 2019

Related U.S. Application Data
(63) Continuation of application No. PCT/US2018/025911, filed on Apr. 3, 2018.
(60) Provisional application No. 62/480,850, filed on Apr. 3, 2017.

(51) Int. Cl. A61B 8/08; A61B 8/00

(52) U.S. Cl. CPC A61B 8/085; A61B 8/4494; A61B 8/5223; A61B 8/485

(57) ABSTRACT

Systems and methods for performing multi-frequency harmonic acoustography for target identification and border detection are described, where a focused confocal transducer having a piezoelectric element and a hydrophone positioned centrally in the piezoelectric element is used. The transducer emits ultrasonic waves toward a target of interest at first and second frequencies. The two waves interfere at a focal plane within the target to generate a third acoustic wave. The target absorbs energy and emits its own unique vibration at the difference frequency (Δf) of the two waves as well as its harmonics. The unique vibration is recorded with a hydrophone, and mechanical properties of the target are ascertained through detection and analysis of the third acoustic wave using a mathematical model implemented by a signal processing circuit.

### CROSS-REFERENCE TO RELATED APPLICATIONS

This application claims priority to, and is a 35 U.S.C. § 111(a) continuation of, PCT international application number PCT/US2018/025911 filed on Apr. 3, 2018, incorporated herein by reference in its entirety, which claims priority to, and the benefit of, U.S. provisional patent application Ser. No. 62/480,850 filed on Apr. 3, 2017, incorporated herein by reference in its entirety. Priority is claimed to each of the foregoing applications.

The above-referenced PCT international application was published as PCT International Publication No. WO 2018/187343 on Oct. 11, 2018, which publication is incorporated herein by reference in its entirety.

### STATEMENT REGARDING FEDERALLY SPONSORED RESEARCH OR DEVELOPMENT

Not Applicable

### NOTICE OF MATERIAL SUBJECT TO COPYRIGHT PROTECTION

A portion of the material in this patent document may be subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

The technology of this disclosure pertains generally to medical imaging, and more particularly to a vibro-acoustography (VA) systems and methods for non-invasive boundary detection of malignant and normal tissues.

### 2. Background Discussion

Changes in elasticity of soft tissues are closely related to tissue pathology. However, estimation of this mechanical parameter for tissue characterization remains difficult in clinical practice. Palpation, the traditional method by which a physician uses touch to sense differences in elasticity between suspicious and normal tissue, requires considerable clinical expertise and is very difficult to utilize when deeper tissues are involved. Early, intra-operative detection and characterization of potential tissue lesions, therefore, specifically calls for more accurate and sensitive diagnostic tools.

In recent years, ultrasonography (US), an acoustic method that measures acoustic wave scattering and impedance differences in tissue, has extensively been used to image and resolve the depth of soft tissue malignancies. US evaluates tumor regions through acoustic wave propagation and tissue scattering that are then correlated to approximate region depth and tissue properties (i.e. malignancies). However, visualization of lesions for surgical resections can only be provided US when prior knowledge of the lesions' location is available. Moreover, US generally underestimates the size of the tumor, which precludes the use of this technology in cases where the tumor tends to be smaller, more well-differentiated, and less palpable.

Other adjunct imaging methods, such as shear-wave elastography (SWE) and optical coherence tomography (OCT), offer high image contrast and resolution, but possess their own limitations. SWE utilizes a focused ultrasound beam to produce a localized radiation stress that perturbs and displaces the target of interest. This perturbation creates deformation propagations in the form of shear waves through the target that are detected by phase-sensitive Magnetic Resonance Imaging (MRI). MRI essentially maps the spatial distribution of the resulting displacement in the target. SWE, however, has limits on the power used for imaging in order to avoid both mechanical and thermal bio-effects, which may cause difficulties in analyzing deeply located regions. Moreover, the generated shear waves are highly attenuated within soft tissue, approximately 2-3 orders of magnitude higher than that of compressive waves, which as a result can limit the depth of penetration. OCT has similarly been used to investigate soft tissue properties and provides spatially resolved information about targeted tissue. However, due to light scattering and attenuation, the depth of penetration and field of view (FOV) of OCT are limited. Additionally, the need for prior knowledge of the target makes standard clinical use of OCT difficult.

Thus, current tumor margin identification methodologies rely on tactile feedback and the experience of the surgeon to decide how much tissue to cut. This method is highly variable in both success and clinical outcome as too little tissue removal can lead to tumor recurrence and too much can lead to debilitating effects.

High incidence of metastases in patients with squamous cell carcinoma (SCC) is very common, and consequently depends heavily on complete surgical resection with negative margins. However, a significant challenge in the treatment is the complexity and the unfamiliarity of the surrounding regions around the malignancy. The primary goal of a surgical treatment is the complete removal of malignant cells while preserving the healthy tissues surrounding it. Thus, there is a pressing need for the use of a real-time, high-resolution imaging modality to accurately differentiate between healthy and malignant tissues. Currently, the most commonly used methods for estimating tumor boundaries are dye injections, conventional ultrasound, manual palpation, and OCT elastography. However, most of these techniques suffer from major limitations such as poor sensitivity and low specificity. Major factors such as contrast, sensitivity, and spatial resolution are critical system parameters for the determination of a boundary for diseased regions with highlighted borders; therefore, there is an acute need for a sensitive yet efficient imaging modality with the ability to rapidly and accurately provide a focal region with accurate boundaries.

### BRIEF SUMMARY

An aspect of the present disclosure is detection of target areas in a body using a technique referred to in this disclosure as vibroacoustography (VA). VA is a non-invasive imaging modality that uses ultrasound-based technology to identify margins between targets with different mechanical properties, using, for example, the viscoelastic (i.e., mechanical) properties of targets to distinguish various material types within an area of interest.

The VA system and methods of the present description improve current ultrasound techniques by providing higher resolution, higher contrast images of various targets at greater depths of penetration. In addition, the VA system and methods of the present description enhance boundary identification making it ideal for intra-operative margin delineation. VA can be used in relative real time in the operating room within the surgical field. This could potentially save a great amount of time and effort from false margins during identification, and can increase the precision of procedures.

The VA system and methods of the present description can be practically and easily implemented in various areas of interest. Alternatives to VA, such as various MR (magnetic resonance) imagery, are relatively expensive and bulky. Real-time CT (computed tomography) has the disadvantage of using radiation for imaging purposes and it provides relatively low tissue contrast. Optical techniques such as OCT (optical coherence tomography) have a small field of view and low depth of penetration compared to VA. Unlike its competitors, VA provides absolute measurements in terms of elastic modulus, density, and viscosity that can quantitatively distinguish regions from one another and allows comparison from patient to patient.

In one embodiment, the VA system the present description comprises a focused confocal transducer or an array of elements, which possess piezoelectric element(s), and a sensitive detector for the acoustic emission and detection. Two pulse generators supply two electrical waves, f₁ and f₂ (where f₂=f₁±Δf) in MHz regime, to the transducer. The electrical waves interfere at a focal plane within the target to generate a radiative acoustic force at the beat frequency in the kHz region of spectrum. As a result of this radiative acoustic force, the target will absorb the energy and will emit its own unique vibration at the beat frequency (Δf) as well as its harmonics which is then recorded by a nearby detector. The acoustic signal that possesses amplitude along with its unique phase then passes through a series of demodulation, and/or filtration, and amplification steps and finally through a lock-in amplifier before signal processing by computer software that incorporates the mathematical model for quantitative target characterization in terms of their mechanical and acoustic properties.

More specifically, the VA system and methods of the present description are directed to characterizing the mechanical properties (e.g., the target's elastic and viscosity properties) through quantitative mathematical modeling. This model allows for absolute quantitative measurement of tissue properties in terms of elastic modulus, bulk modulus, shear modulus, shear velocity, density, and viscosity.

The sensitivity and resolution performance limits of the vibroacoustography (VA) system were evaluated. A VA transducer with confocal geometry was utilized and its feasibility in imaging targets was evaluated. The beam profiles of the emitted radiation amplitude for a point target, a 1 mm stainless steel bead embedded in 15% gelatin TMP, on the lateral and axial planes, were derived and validated by the experimental findings. Moreover, the system sensitivity and specificity in distinguishing regions with different mechanical properties in line-pair tissue-mimicking phantoms (TMPs) were examined. Two-layered TMPs of same material type but differing concentration, and three and four-layered TMPs of a combination of agar and gelatin concentrations were imaged using our VA system. The results illustrate that the VA system is sensitive in boundary detection of both homogeneous materials with dissimilar concentrations, as well as heterogonous materials. Both amplitude and phase images demonstrated our VA imaging system's ability to image multiple-layered TMPs by providing detailed information about the target; thus, for future in vivo experimentation, both images should be utilized for accurate tissue boundary delineation. Moreover, the fabrication, alignment, and design of the confocal transducer are other system parameters that may be modified to optimize the VA system, and the beam scanning rate may be increased for clinical use of VA. The theory and experimental techniques disclosed herein may also be used for beam forming design and system evaluation for various applications of VA.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWING(S)

The technology described herein will be more fully understood by reference to the following drawings which are for illustrative purposes only:

FIG. 1 shows a schematic diagram of a vibro-acoustography system in accordance with the present description.

FIG. 2 shows a plan view of the confocal curved element of FIG. 1.

FIG. 3 shows high-level a schematic diagram of a VA system with processing components.

FIG. 4 shows a normalized transverse beam profile at Δf=38 kHz for the confocal transducer.

FIG. 5 shows the normalized axial beam profile Δf=38 kHz for the confocal transducer.

FIG. 6A and FIG. 6B show normalized lateral (FIG. 6A) and axial (FIG. 6B) modulation transfer function (MTF) as a function of spatial frequency at Δf=38 kHz for a confocal transducer.

FIG. 7A and FIG. 7D illustrate visible images of three-layered and four-layered phantoms, respectively. FIG. 7B and FIG. 7E illustrate three-layered and four-layered phantoms, respectively. FIG. 7C and FIG. 7F illustrate the phase image of the three-layered and four-layered phantoms, respectively.

FIG. 8 is a schematic diagram of a spherical-tip micro-indentation experimental set-up in accordance with the present description.

FIG. 9A through FIG. 9C are elastic relaxation behavior plots of agar, gelatin, and PVA TMPs, respectively, as a function of time at in indentation of 600 μm.

FIG. 10A and FIG. 10B are relaxation behavior plots of ex vivo rat and porcine animal tissues.

FIG. 11 is a plot of acoustic outflow of biological tissues, specifically adipose, soft tissue, and muscle, as a function of resonant beat frequency.

FIG. 12 shows a plot of a steady state response of a vibroacoustography signal.

FIG. 13 shows the transient response of tissue to initial vibracoustic excitation.

### DETAILED DESCRIPTION

Herein, we evaluate the sensitivity and resolution performance limits of a VA system in: 1) Two-layered and multiple-layered tissue-mimicking phantoms (TMPs) for sensitivity and specificity limits, and 2) TMP with an embedded stainless-steel bead for resolution limits. Line-pair phantoms composed of agar and gelatin, n=1 for each experiment, were imaged to generate the point spread function (PSF) and sensitivity of the VA system. These results may further support VA as an accurate and sensitive tool for in vivo tumor boundary detection during intra-operative surgical procedures.

Described herein is an imaging modality, referred to herein as vibro-acoustography (VA), that generates a map of the mechanical response of a target to a dynamic acoustic radiation force. In response to the applied oscillating force, the object vibrates and produces an acoustic emissions field at the difference frequency (Δf). This emissive wave is collected from the object using a highly sensitive acoustic hydrophone. An image is created of the spatially varying signal, particularly amplitude and phase, of the target by scanning the focused beam spot over the object.

1. System Configuration

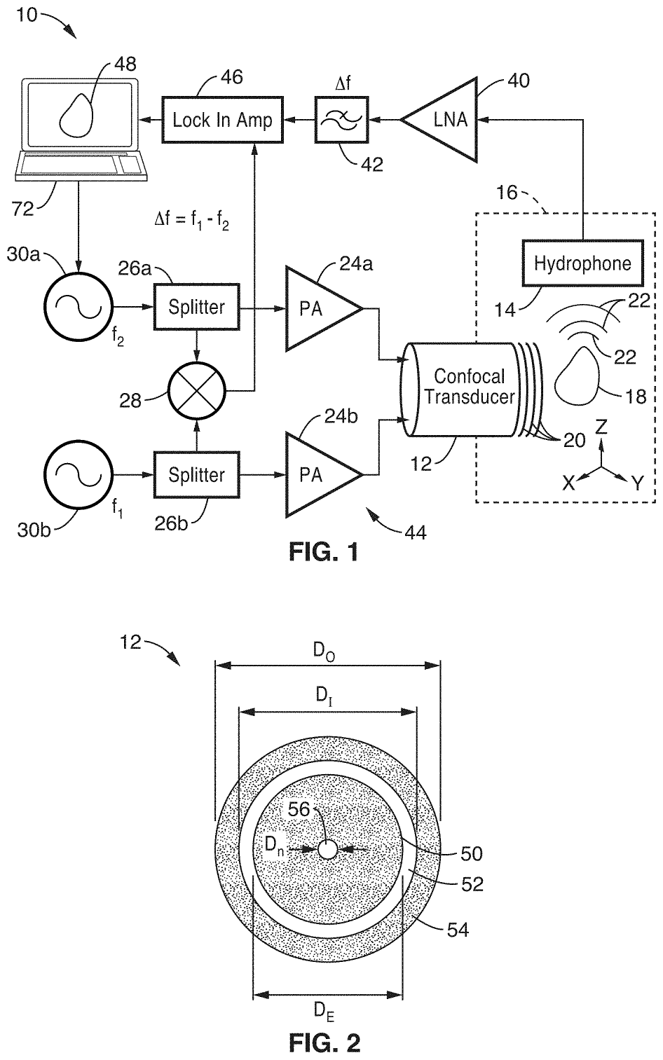

Referring to FIG. 1 through FIG. 3, one embodiment a VA system 10 according to the presented technology includes a focused confocal transducer 12 (Boston Piezo-Optics, Bellingham, Mass.) and a compact hydrophone 14 for detection. Two pulse generators 30a and 30b supply two electrical sinusoidal waves, f₁ and f₂ (where f₂=f₁+Δf), to the transducer 12. Waves f₁ and f₂ are applied to the inner 50 and outer portions 54 of the piezoelectric element 12 (see FIG. 2) to emit two distinct acoustic waves f₁ and f₂ as waves 20 into the tissue 16 at the focal plane of the transducer 12 (e.g., about 5.9 cm). The two waves interfere at the focal plane within tissue or material 16 to vibrate the tissue 16, generating a third acoustic wave 22 in the kHz region of spectrum. In the test setup of the present description, the tissue 16 is simulated via tissue mimicking phantoms (TMPs), which are placed in a water tank to achieve minimal signal loss as the acoustic wave propagates. This energy transformation is accomplished as the target tissue 18 (e.g. TMP) absorbs the energy and emits its own unique vibration at the difference frequency (Δf), as well as its harmonics, which is then recorded by the nearby compact hydrophone 14.

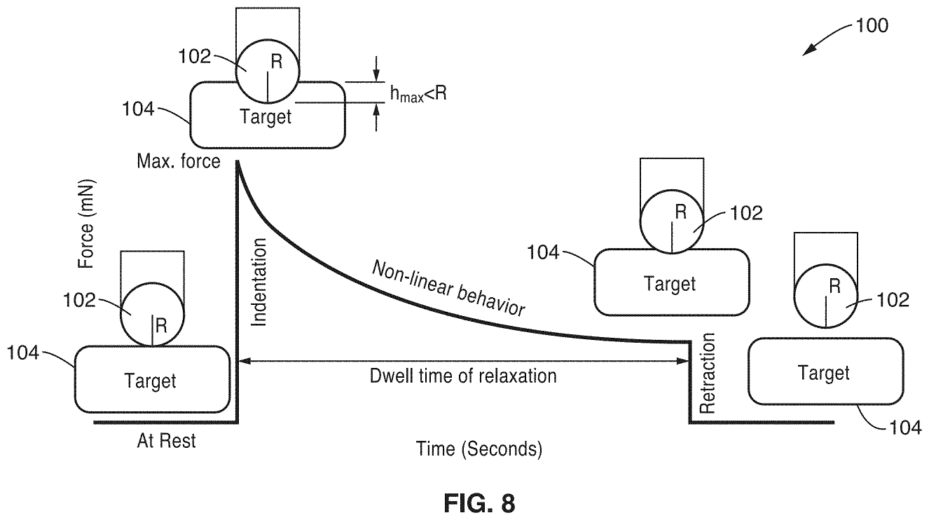

The signal received by the hydrophone 14 is then filtered and amplified with a Low Noise Amplifier (LNA) 40 and programmable filter 42 (e.g. SRS 650, Stanford Research Systems, Inc. Sunnyvale, Calif.), and then passed through a lock-in amplifier 46 (e.g. SRS 844, Stanford Research Systems, Inc. Sunnyvale, Calif.), and processed by a signal processor 72 (e.g., computer hardware processing circuit 78 and related software instructions 74, see FIG. 8) for absolute characterization. Detecting the acoustic responses not only generates contrast sufficient for image formation, but the acquired data enables for the quantitative characterization of material properties without reliance on reflection/attenuation of acoustic waves.

In a preferred embodiment, system 10 generates two unmodulated continuous wave (CW) ultrasonic beams (f₁ and f₂) at slightly different frequencies in the low-MHz range to impose a low frequency kHz stress field (or beat frequency) 22. Each beam is generated by a coherent function generator (30a and 30b), e.g. Agilent 33220A, Santa Clara, Calif. and power amplifier (PA) 24a and 24b, e.g. AR Modular, Inc. Bothell, Wash. The two amplified frequencies are then fed into a confocal transducer 12 with f₁ coupled to the inner transducer ring 50 and f₂ coupled to the outer ring 54. This produces two converging beams that overlap at the target of interest 18. Depending on the viscoelastic properties of the target 18, the generated radiation force will cause a portion or the entire tissue of interest to vibrate at the difference frequency Δf=|f₁−f₂|. Further, the combination of viscoelastic properties and tissue volumes result in mechanical non-linearities that describe the harmonic generation behavior. The result is a variation on the acoustic yield of the harmonics of Δf. The presence and relative strengths of these harmonics form a unique tissue type identifier.

The acoustic harmonic emission of the tissue is detected by a hydrophone 14 (e.g. TC4014-5, Teledyne Reson Inc. Goleta, Calif.) located near the illuminated tissue.

In a preferred embodiment shown in FIG. 2, the hydrophone 14 is disposed within a hole 56 at the center of the inner transducer element 50. While hole 56 is shown in the center of the inner transducer, it is appreciated that it may be positioned at any location within the device. In one configuration, the outer diameter Dₒ of the outer transducer 54 is 45.04 mm, with an outer diameter D_{I} of 30.86. The diameter D_{E} of the inner transducer is sized smaller than D_{I} (e.g. 28.56 mm) such that a gap 52 is formed between inner 50 and outer 54 transducers. Center hole 56 is sized (e.g. 2.7 mm) to receive hydrophone 14.

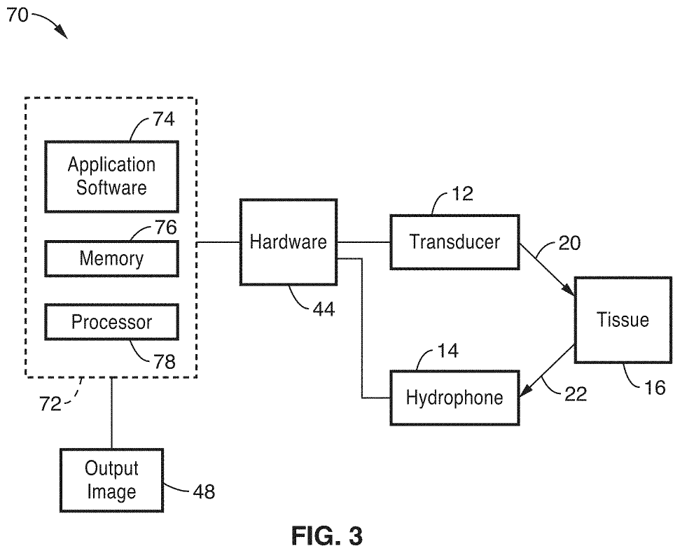

FIG. 3 shows high-level a schematic diagram of a VA system 70 with processing components. System 70 comprises a computing device 72 configured for executing application software 74 that is stored in memory 76 via a processor 78. Application software 74 may be configured for controlling transducer 12 and hardware 44 for generating the waves f₁ and f₂ (20) into tissue 16. Software 74 also may be configured to receive the output signal of hydrophone 14 (from waves 22), and process the signal to generate output image 48.

Hardware 44 further comprises a pair of splitters 26a and 26b (e.g. 3 dB splitter (Minicircuits, Inc. Brooklyn, N.Y.)) that split the signals from signal generators 30a and 30b. Part of the signals are sent to a mixer 28 (e.g. Minicircuits, Inc. Brooklyn, N.Y.), the output of which is fed to lock in amp 46 (e.g. SRS 844, Stanford Research Systems, Inc. Sunnyvale, Calif.) as a reference signal for lock-in and it goes through, wherein band pass filter is used in conjunction with output from mixer 28 to remove difference frequency (f1 and f2) from the LNA processed data 42 received from hydrophone 14. The application software 74 acts as a phase-sensitive spectrometer to detect the output signal. In a preferred embodiment, the images are generated by raster scanning the beam 20 throughout the field of view of tissue 16 through mechanical scanning or beam steering means (not shown), and processing the scanned data with application software 74 to generate an image map 48 of the mechanical (e.g. viscoelastic) response of the target 18 to the acoustic radiation force 20. Application software 74 may be configured so that pixel values are computed as the power at a particular harmonic or algebraic combination of powers at multiple harmonics.

In one embodiment, the application software 74 is configured to use acoustic as well as mechanical properties of the target tissue, such as elasticity and viscosity, which are not limited by the boundaries of the generated acoustic waves, and can provide absolute quantitative measurements of the target tissue. Application software 74 may further include a mathematical model based on the geometry, mechanical properties, and acoustic properties of the tissue in the phase and amplitude measurement to extract quantitative information from target.

In one embodiment, the curved confocal transducer (Boston Piezo-Optics, Bellingham, Mass.) has a radius of curvature (ROC) of ˜60 mm, to cause an interference at the focal region, which is meticulously placed on the target 18. The confocal transducer 12 may be constructed by dividing the back electrode of the piezoelectric ceramic into an inner and outer ring, such that the elements have identical beam axes and focal lengths. The two elements are excited by two slightly different frequencies, f₀=3.16 MHz and f₀+Δf=3.198 MHz, in continuous wave (CW) form. The pressure field along the z-axis is modeled by:

$$\begin{matrix}p ( z , t ) = {P}_{1} ( z ) cos ( 2 π {f}_{1} t + {ϕ}_{21} ( z ) ) + {P}_{2} ( z ) cos ( 2 π {f}_{2} t + {ϕ}_{22} ( z ) ) & Eq . 1 \\ where & \\ {P}_{1} ( z ) = {Zu}_{0} \frac{R}{\vert z \vert} \vert 1 - exp ( \frac{j π {a}_{1}^{2}}{{λ}_{1} R} \cdot \frac{z}{z + R} ) \vert & Eq . 2 \\ and & \\ {P}_{2} ( z ) = {Zu}_{0} \frac{R}{\vert z \vert} \vert 1 - exp ( \frac{j π {a}_{21}^{2}}{{λ}_{2} R} \cdot \frac{z}{z + R} ) - exp ( \frac{π {a}_{22}^{2}}{{λ}_{2} R} \cdot \frac{z}{z + R} ) \vert & Eq . 3\end{matrix}$$

P₁(z) and P₂ (z) are amplitude functions, and f₁ and f₂ are frequencies in MHz. Z=ρc is the acoustic impedance, where ρ and c are the density and propagation speed, respectively. λ₁ and λ₂ are ultrasound wavelengths, u₀ is the amplitude of the particle velocity at the transducer surface, and a is the transducer radius. φ₂₁(z) and φ₂₂ (z) are phase functions and R is the ROC. The beam was raster scanned over the target surface at Δf=38 kHz. At each point, the peak amplitude and the phase of the emission were measured and used to generate an image. Each pixel value in the image was scaled to the maximum value of the peak amplitude and phase from the target. The scanning resolution is 0.04 mm for the lateral scan (x and y axes) and 2.0 mm for the axial scan for the first part of the study. All units are in the MKS system.

The oscillatory acoustic radiation force on a point target at (x₀,y₀) on the focal plane is given by Eq. 4 and the oscillatory radiation force on a unit target at zₒ on the transducer axis is illustrated in Eq. 5:

$$\begin{matrix}{F}_{Δω} ( {x}_{0} , {y}_{0} ) = \frac{1}{{c}^{2} ρ} {P}_{1} ( {r}_{0} ) {P}_{2} ( {r}_{0} ) cos ( Δω t + {ϕ}_{12} - {ϕ}_{11} ) = \frac{ρ {u}_{0}^{2} {π}^{2} {a}_{1}^{2}}{{λ}_{1} {λ}_{2} {R}^{2}} jinc ( \frac{{r}_{0} {a}_{1}}{{λ}_{1} R} ) [ {a}_{22}^{2} \cdot jinc ( \frac{{r}_{0} {a}_{22}}{{λ}_{2} R} ) - {a}_{21}^{2} jinc ( \frac{{r}_{0} {a}_{21}}{{λ}_{2} R} ) ] cos ( Δω t + {ϕ}_{12} - {ϕ}_{11} ) & Eq . 4 \\ {F}_{Δω} ( {z}_{0} ) = ρ {u}_{0}^{2} \frac{{R}^{2}}{{z}_{0}^{2}} \vert 1 - exp ( \frac{j π {a}_{1}^{2}}{{λ}_{1} R} \cdot \frac{{z}_{0}}{{z}_{0} + R} ) \vert \times \vert 1 - exp ( \frac{j π {a}_{21}^{2}}{{λ}_{2} R} \cdot \frac{{z}_{0}}{{z}_{0} + R} ) - exp ( \frac{j π {a}_{22}^{2}}{{λ}_{2} R} \cdot \frac{{z}_{0}}{{z}_{0} + R} ) \vert cos ( Δω t + {ϕ}_{12} - {ϕ}_{11} ) & Eq . 5\end{matrix}$$

While a configuration with a single confocal transducer 12 has been described, it will be appreciated that a plurality of transducers could be employed as well. For example, alternative embodiments may comprise linear or annular arrays of piezoelectric elements that will transmit and receive the radiation acoustic force. Whether a linear or a curved array would be used would depend on the particular application. Furthermore, the number of elements in an array can vary depending on the application, such as arrays of 64 or 128 elements.

In the embodiments comprising arrays, two pulse generators would supply two electrical waves, f₁ and f₂ (where f₂=f₁±Δf), to the array of elements. The low MHz ultrasonic tones would travel to the piezoelectric elements and emit two distinct acoustic waves at the focus plane of the array. The two waves would interfere at a focal plane within tissue or material to generate a third acoustic wave in the kHz region of spectrum. This energy transformation is accomplished as the target absorbs the energy and emits its own unique vibration at the difference frequency (Δf) as well as its harmonics. After detection, the signal would pass through a series of demodulation, filtration, and amplification steps before it is processed by computer software for absolute characterization using a mathematical model based on the mechanical properties of the target. Detecting the acoustic responses not only generates contrast sufficient for image formation, but the acquired data enables for the quantitative characterization of material properties.

In further embodiments of the arrayed configurations, control software implemented in application software 74 that is employed for electronic steering of the elements in the array. For example, elements could be driven in pairs and the specific pairs selected depending on how the beam is steered.

a. Image Generation

Imaging systems are typically characterized by resolution performance limits, which can be studied through the point spread function (PSF) and modulation transfer function (MTF). PSF relates a generated image of a target to its physical (i.e. actual) appearance. Image formation in VA is based on the acoustic emission of the target in response to an acoustic radiation force perturbation. The emission is a function of the dynamic component of the radiation force and the object's physical characteristics, such as size, shape, mechanical properties, and geometry. To calculate the PSF of VA system of the present description, we consider a point source with a unit mechanical response at position (x₀, y₀), analogous to an impulse response in the XY plane. A point source is used because it is represented in a single pixel as an “ideal” image. However, using the VA system of the present description, it will be portrayed as more than one pixel, a “real” image. We rationalize the PSF of the imaging system as:

g(x,y)=h(x,y)*f(x₀,y₀) Eq. 6

where g(x,y) is the output image, and h(x,y) is the PSF convolved with the input object, f(x₀,y₀).

Modulation transfer function, MTF, is another way to measure the frequency response of the system and to provide the spatial resolution response of an imaging system. MTF demonstrates the system's ability to transfer contrast to a resolution from the target to its image. Moreover, MTF is another way to incorporate resolution and contrast into a single parameter. While PSF represents system performance in the spatial domain (mm), MTF represents it in the frequency domain (mm⁻¹). They can further be related by the Fourier transform:

MTF= [symbol] (PSF) Eq. 7

where [symbol] represents the Fourier transform.

For image acquisition, a Lock-in Amplifier 46 was used to detect the amplitude and phase of the output acoustic emission that was collected by the acoustic hydrophone 14 and filtered by the programmable filter 42. The point source target 18 used for the first part of the resolution testing was a 1 mm stainless steel bead embedded in a 15% (% weight) gelatin TMP, measuring 20×20×30 mm³. The phantom was submersed in an ultrasonic testing water tank and secured by 4 Teflon screws in a sample holder. 15% gelatin was utilized as an accurate analog to human tissue, particularly of the head and neck, due to the nearly identical acoustic properties between the soft tissue and the TMP.

Based on the two high ultrasonic frequencies used, the minimum spot size of the VA system is predicted to be slightly larger than 500 μm, which is sufficient to directly measure the PSF. Additionally, the elastic modulus of stainless steel is orders of magnitude higher, thus providing efficient conversion from high frequency to difference frequencies. Therefore, due to the elastic properties of the sphere, an ideal emissive signal will be detected with sufficient contrast from the background, 15% gelatin phantom. Spot sizes, defined as full width half maximum (FWHM) about the peak signal, were calculated from the scans. The bead in the phantom was imaged and its effect on the beam geometry (i.e. spot size) was ascertained. A similar set-up was used for the second part of this paper: Evaluation of the VA system's sensitivity and specificity in line-pair phantoms.

Tissue-mimicking phantoms (TMP's) provide ideal tissue models, and can be constructed with well-defined dimensions and acoustic properties to reduce potential confounding variables during imaging. Moreover, phantoms used for ultrasound imaging modalities possess acoustic properties, such as characteristic acoustic impedance, attenuation, backscattering coefficient, and compressional and shear wave speeds of sound, near those of the mimicked tissue.

Agar blocks (Agar, Sigma-Aldrich St. Louis, Mo.) of differing water/agar concentrations were fabricated. These blocks of agar, containing 2%-4% agarose (% weight), were mixed and heated above 90° C. to maximize cross-linking between polymers. Increasing the amount of agarose in the mixtures leads to a corresponding increase in the elasticity of the phantom. Gelatin blocks (Porcine Gelatin, Sigma-Aldrich St. Louis, Mo.) possessing water/gelatin concentrations of either 10%-20% (% weight) were used as a second type of tissue substrate. To remove air bubbles from the final mixture, each gelatin solution was placed in a centrifuge at a rotation speed of 2 rcf (relative centrifugal force) for a period of 30 seconds. Increasing the gelatin concentration also increases the elasticity of the phantom.

For the evaluation of our system's axial and lateral resolution, a 20×20×30 mm³ 15% (% weight) gelatin TMP with an embedded 440C grade 24 stainless steel bead (NEMB, Norfolk, Conn.) with 1 mm diameter was used. The bead was embedded ˜9 mm into the gelatin phantom and was raster scanned in the XY and XZ planes multiple times.

For sensitivity and specificity evaluation of VA system of the present description, line-pair phantoms were used. Two-layered line-pair phantoms of either agar or gelatin were synthesized to evaluate the ability of the VA system in distinguishing boundary regions in two-layered, isotropic targets. These two-layered line-pair phantoms were created from one type of material (i.e. agar or gelatin), but were split into two regions of different concentrations with varying widths to simulate proximate, homogeneous tissue regions. 30×30×30 mm³ phantoms of 1.) 10% and 20% gelatin and 2.) 2% and 4% agar phantoms were chosen for the first part of this study. These TMP concentrations and types were chosen because of the elastic modulus difference between them: 10% and 20% gelatin ˜65 kPa and 2% and 4% agar ˜85 kPa. These significant differences in mechanical properties make these TMPs ideal candidates for sensitivity and specificity evaluation of the VA system. Additionally, these particularly types of TMPs were synthesized due to their similarity to human soft tissues (i.e. prostate, breast, liver, and head and neck) in terms of acoustic properties such as speed of sound, density, and signal attenuation. The VA signal analysis of these two-layered TMPs will provide a good proof of concept for potential applications in medical imaging.

The second part of the study utilized multiple-layered (i.e. agar and gelatin) line-pair phantoms of varying layers to emulate heterogeneous areas of human tissue. The multiple-layered phantoms were fabricated to investigate the sensitivity and specificity of the VA system of the present disclosure for in vivo tissue identification and border detection by combination of agar and gelatin regions into one phantom. We synthesized one three-layered phantom, 35×35×35 mm³, with a region of 2% agar sandwiched between two regions of 10% gelatin and 15% gelatin with different widths. The three-layered phantom possesses both high and low differences in elastic modulus, which help capture the full scope of sensitivity detection limits of the VA system. The difference in elastic modulus in the three-layered phantom are ˜55 kPa between 10% and 15% gelatin, ˜10 kPa between 15% gelatin and 2% agar, and ˜75 kPa between 10% gelatin and 2% agar.

Additionally, one four-layered phantom, 25×25×25 mm³, with alternating regions of 3% agar and 20% gelatin, was synthesized. The elastic modulus difference between 3% agar and 20% gelatin is ˜50 kPa, creating distinct viscoelastic contrast, and hence making the four-layered phantom suitable for VA system sensitivity evaluation. The mentioned concentrations were used to mimic the heterogeneous environment common to in vivo settings during intra-operative procedures. These phantom sizes were chosen to stabilize the structural integrity of the phantom during the synthesis, fabrication, and imaging process.

b. Results

The VA system's performance in imaging targets and boundaries within the FOV were evaluated. To first assess the resolution of the VA system 10, we evaluated the normalized transverse amplitude component of the collected signal from the 1 mm diameter stainless steel bead embedded in the 15% gelatin phantom, at Δf=38 kHz. The acoustic difference frequency generated from the target under test is detected by a near-by hydrophone. The signal was bandpass filtered by a programmable bandpass filter, with a bandwidth of 10 kHz, and a Lock-in Amplifier, with reference signal of 38 kHz. Each raster scan was taken in a step size of 0.4 mm in the lateral direction and 2 mm in the axial direction. The target was positioned 59 mm away from the transducer. MATLAB was utilized to calculate the average dBm value of a 3×3 square of pixels centered around the bead, corresponding to an area of 1.2×1.2 mm² in the actual phantom; this large scan area was used to avoid any conforming error due to translating stage or phantom movement between multiple axial scans in the ultrasonic test tank.

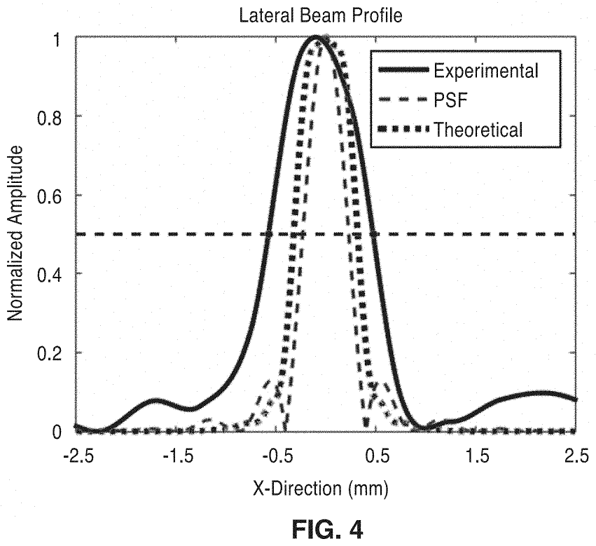

FIG. 4 displays the theoretical and experimental results of the lateral beam profile of the transducer's confocal geometry. The large-dashed line delineates the calculated PSF. Given the curved geometry of the transducer, the stress field is evidently symmetrical FIG. 3, the theoretical curve (small-dashed line) the convolution of the confocal transducer's PSF with an ideal profile with width of 1 mm, which is analogous to the sphere's diameter. The experimental graph (solid line) illustrates the beam profile in x-axis of the same embedded 1 mm diameter stainless steel bead. The theoretical and experimental plots of acoustic emission of the bead with a finite size, 1 mm diameter, appear to agree. The calculated FWHM (horizontal line) conveys that the VA system 10 measured the bead's diameter to be 1.05 mm. The actual and measured value of the bead's diameter are very close, ˜4.9% difference, which demonstrates the system's high-resolution performance.

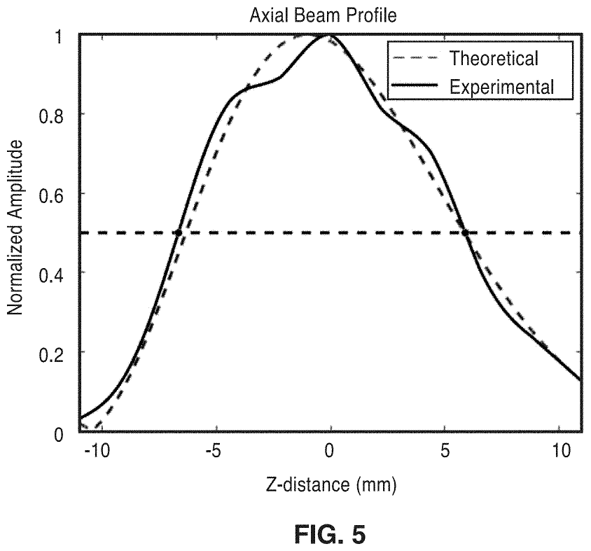

FIG. 5 shows the normalized amplitude plot of the axial beam profile as a function of axial distance. The solid line represents the experimental axial profile in the z-direction of a 1 mm stainless steel ball embedded in 15% gelatin phantom cube. The dashed curve represents its theoretical axial resolution. The horizontal dashed line denotes the FWHM of the experimental axial beam profile. The calculated axial resolution, illustrated by the dashed line, at FWHM was 12.5 mm, which is very close to the reported literature value, 12.2 mm. Although the lateral profiles were relatively close, the overall axial resolution profile of the power field is somewhat different from the theoretical expectation. This deviation can be due to the transducer's construction, such as alignment fabrication errors in the two confocal parts. Another possible source of error can occur from the presence of the transducer's side lobes. In most confocal transducers, beam interference only occurs at the focus region where the two beams meet. Thus, the length of the focal region and the generation of side lobes can be potential reasons for the discrepancy in the results. Additionally, any other signal (i.e. mechanical motors on the tank) with a frequency that falls within the bandwidth of the receiver can cause interference with the collection scheme and may reduce the signal to noise ratio (SNR).

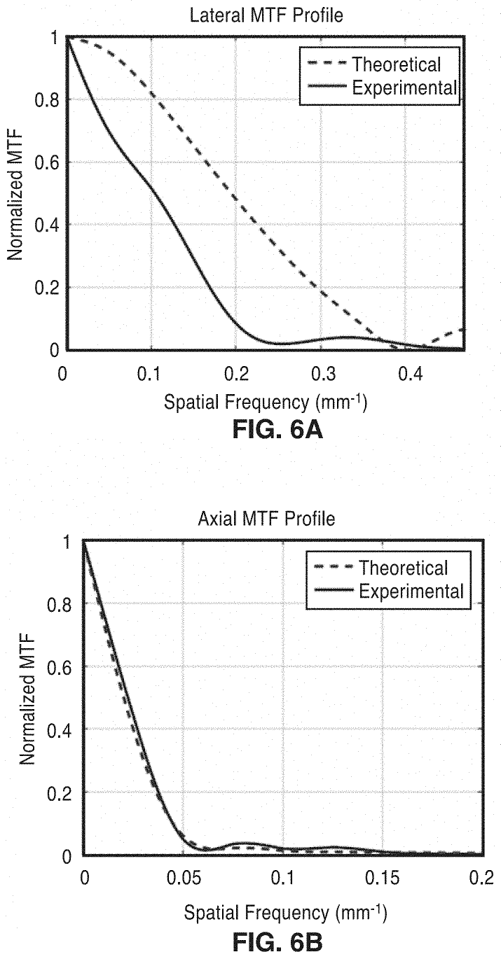

Moreover, another way to interperate the system's resolution performance limit is through the MTF. As apparent in FIG. 6A, the axial MTF of the experimental plot closely matches to that of the theoretical. However, for the lateral profile shown in FIG. 6B, theoretical plot contains more of the signal as the spatial frequncy increases as apposed to the experimental plot.

Since the MTF is typically calculated from the PSF, as the width of the beam profile decreases, the corresponding values in the MTF plot will fall to higher values at the same spatial frequency, which is evident in the comparison between axial and lateral MTF profiles. The cutoff spatial resolution is usually considered to be the frequency at which the MTF crosses the 10% level. Thus, as depicted in FIG. 6A, the cutoff frequency for the axial MTF profile is ˜0.045 mm⁻¹ for both theoretical and experimental, but ˜0.34 mm⁻¹ and ˜0.19 mm⁻¹ for theoretical and experimental lateral MTF profiles, respectively.

Both experimental and theoretical axial MTF profiles have roughly the same behavior and cutoff frequency. However, this is not the case for the lateral MTF profile. Since the lateral PSF is much smaller than the axial PSF, the behavior, including cutoff frequency, of experimental and theoretical lateral MTF profiles differ more than the axial MTF profiles. This likely resulted from the small FWHM in the PSF in the lateral direction.

The VA system's sensitivity in differentiating regions of varying concentrations of the same material was also determined. The phantoms were imaged using the VA system 10 and laterally scanned only in one XY plane. The targets were imaged in 0.5 mm step size at the focus plane of the confocal transducer 12. The mean power and lateral distance of each region in the two-layered TMPs were calculated using MATLAB. Table 1 and Table 2 display the mean power (dBm) and lateral distance (mm), both empirically measured and theoretically calculated, for the two regions in each line-pair phantom. A ruler was used to measure the lateral distance of each region prior to imaging, and the measurements were compared to the VA-measured lateral distance.

Table 1 shows results for Agar two-layered phantoms, possessing one layer of 2% and another of 4%. Table 2 shows results for Gelatin line-pair phantoms, possessing one layer of 10% and another of 20%, results are presented.

The line-pair TMPs for both agar and gelatin followed inverse trends between concentration (i.e. elasticity) and average signal power. The signal power appeared to be higher for the lower concentration of each two-layered line-pair phantom. This result is believed to be due to the elasticity differences between the two regions in each line-pair phantom. 2% agar and 10% gelatin have the lower elastic modulus in each of their respective line-pair phantoms, and thus are more easily perturbed by an ultrasonic wave from the VA system 10. This lack of stiffness induces an increased magnitude of emission from the lower concentration portion of the line-pair phantom, resulting in a higher mean power for the lower concentration in each line-pair phantom. In contrast, a lower mean power is observed in the stiffer, higher concentrated portion of each phantom.

Although proximate regions in each line-pair phantom can be definitively distinguished based on mean power, we also sought to quantify the lateral distance of each region using our VA-generated images. Our VA-calculated lateral distances fell within ±1 mm of the measured (actual) lateral distances obtained prior to imaging. The minimal differences between calculated and measured lateral distance reinforce the potential of the VA system 10 for acute boundary detection.

After establishing the VA system's efficacy and sensitivity in two-layered line-pair phantoms, similar experiments were conducted on three-layered and four-layered phantoms. This was done to more accurately simulate the complexity and variety of human soft tissue during intra-operative surgical procedures.

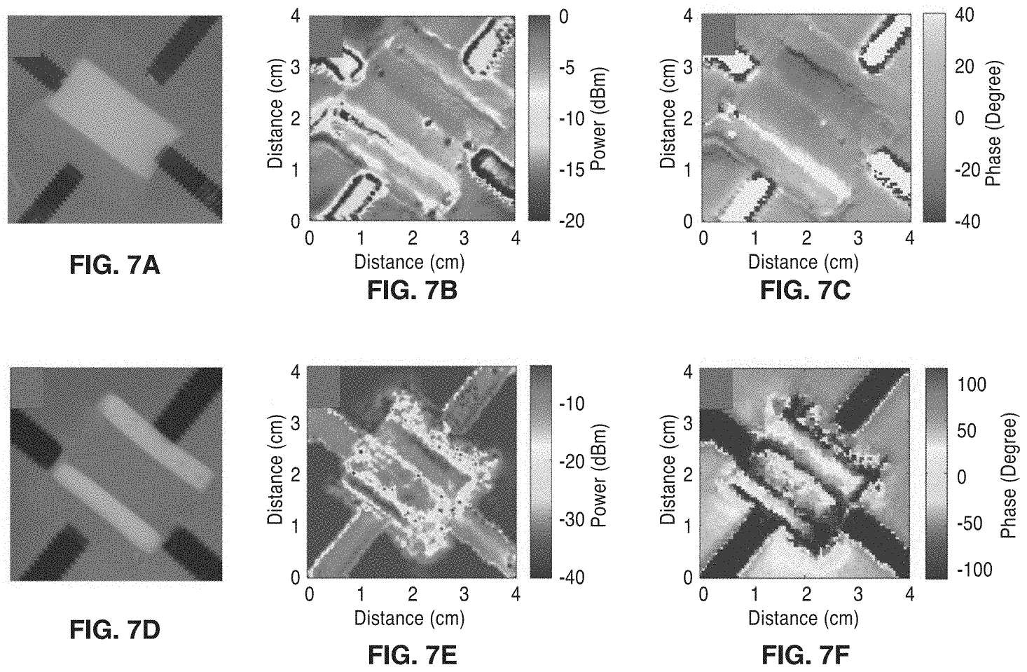

The phantoms were imaged using the VA system 10 with a similar set-up as the two-layered TMPs. The average power and lateral distances of regions with differing concentrations were calculated using MATLAB. FIG. 7A through FIG. 7F show the acquired amplitude and phase images of three-layered and four-layered TMPs. Specifically, FIG. 7A and FIG. 7D display the visible images of the three-layered and four-layered phantoms, respectively. FIG. 7B and FIG. 7E display the amplitude of the detected signal, and FIG. 7C and FIG. 7F display the phase image of each phantom.

Table 3 shows the average power and the measured and calculated lateral distance for each region of the multiple-layered phantoms. The first region listed is at the bottom of the TMP and the last region is at the top of the phantom shown in the visible images (FIG. 7A and FIG. 7D). A ruler was again used to measure the lateral distance of each region and the results were compared to the VA-calculated lateral distances generated using MATLAB.

Upon visible inspection of FIG. 7A through FIG. 7F the VA system 10 can clearly distinguish between agar and gelatin phantom types by detecting the boundaries of each region. The amplitude image of the three-layered phantom (FIG. 7B) and the phase image of the four-layered phantom (FIG. 7F) illustrate the best contrast between different phantom regions. However, the contrast in FIG. 7C through FIG. 7E is not as clear. The presence of undefined boundaries in the amplitude image of the four-layered phantom (FIG. 7E) could be attributed to similarities in the emitted acoustic power between 3% agar and 20% gelatin. On the other hand, the inability to detect the phase changes at the boundary of the 2% agar and 10% gelatin may account for the deficient contrast in the phase image of the three-layered phantom (FIG. 7B). Both the amplitude and the phase images have valuable information about the target, and thus both images should be considered to provide improved contrast in VA imaging.

Another important factor that was examined included the relative difference in mechanical properties of the line-pair phantoms. The difference in elastic modulus between each region in both multiple-layered phantoms were examined by comparing the detected emissive acoustic signal. In instances of both large (˜50 kPa) and small (˜10 kPa) differences in elastic modulus, a clear boundary in both amplitude and phase images were apparent. Moreover, in the case of the four-layered TMP, the mean power difference between identical regions of 20% gelatin and 3% agar were approximately the same (±1.5 dBm). Similar to trends that were previously observed, softer regions were characterized by higher acoustic signals compared to those of stiffer regions. In the case of the three-layered TMP, the trend between 10% gelatin and 15% gelatin portions showed an inverse relationship between stiffness and amplitude. In contrast to this, the 2% agar region of the three-layered TMP did not illustrate the lowest signal as originally expected. Agar 2% possesses the highest elastic modulus of the three layers, and is thus hypothesized to emit the lowest amplitude response in a VA scan. This discrepancy may be due to the orientation of the phantom; since the 2% agar layer is situated between two gelatin layers with lower elastic moduli, some of the generated acoustic signal could have been affected by the neighboring layers, causing interference. This variable must be evaluated in further studies by varying the thickness and concentration of each layer in the TMP.

The VA-generated images of the TMPs generate high relative SNR with their respective background; however, within the TMPs, there were a few regions with speckles that may cause discrepancies. First, in VA imagery of the three-layered TMP, a semi-superficial line in the middle of the 3% agar layer appeared. This artifact likely resulted from the fabrication process due to a use of a divider in making the phantom. Second, the transducer itself generates a sound field, different from the two high frequency tones, that interferes with the acoustic emission of the object. Lastly, air bubbles that form on the ground surface of the transducer during scanning and defects that occur in the TMP fabrication process are other potential factors that need to be considered as sources of noise in VA imagery. The manner in which the phantom is secured during scanning is another possible source of noise. Teflon screws were used to secure the TMP, and such screws can exert external forces onto the phantom that can either attenuate or cause interference in the acquired signal.

Another examined parameter in the line-pair TMPs was the lateral measurements. VA-calculated lateral distances were close to the measured (actual) distances; each length differed by no more than 1 mm in every data set. This result illustrates the high precision of VA in boundary detection of proximate regions within a section of a multiple-layered TMPs. The high efficacy of experimental distance measurement coupled with high image contrast, based on mechanical properties (i.e. elastic modulus), makes VA a promising technique for further use in intra-operative surgical resection procedures.

The above findings have important implications. The VA system 10 can not only distinguish between two proximate material types but also identify regions with different concentrations of the same material. These system capabilities will be essential for future clinical use of VA in an intra-operative setting. In a hypothetical situation where surgeons are placed in similar scenarios in which healthy and abnormal tissue are positioned next to each other, and the abnormal tissue must be excised from the area, the VA system 10 may be helpful in the determination of clear boundaries between specified regions.

3. Quantitative Characterization of Viscoelastic Behavior in TMP's and Ex Vivo Animal Tissues

The following description investigates viscoelasticity theory and provides viscoelastic experimental results obtained in both tissue-mimicking phantoms (TMPs) and ex vivo tissue. A spherical-tip micro-indentation technique along with the Hertzian model are employed to acquire absolute, quantitative, point measurements of the elastic modulus (E), viscosity (1r), and time constant (T) in isotropic homogeneous TMPs and ex vivo hepatic tissue in rat and porcine models. Liver elastic moduli for porcine liver, porcine gallbladder, and rat liver were 2.55±0.09 kPa, 4.7±0.7 kPa, and 2.76±0.09 kPa, respectively. The viscosity values for the same respective tissues were 0.135±0.006 MPa·sec, 0.18±0.03 MPa·sec, and 0.147±0.007 MPa·sec. Statistically significant viscoelastic differences between porcine liver and bile duct tissue convey that imaging modalities which utilize the mechanical properties of tissue as a primary contrast mechanism, such as vibroacoustography and elastography, can potentially be used to differentiate between proximate organs in a clinical setting. These viscoelasticity results may facilitate more accurate tissue modeling and add information not currently available to the field of systems characterization and biomedical research.

In the following description, the VA system 10 is investigated for accurate modeling of viscoelastic properties of tissues. Rheological models, specifically the Hertzian model, are used to characterize mechanical properties of pre-clinical targets. This model was used in conjunction with a micro-indentation technique that uses a rigid, spherical-tip to acquire absolute measurements of mechanical properties on TMPs and ex vivo porcine and rat hepatic tissues. Elastic modulus, viscosity, and time constant results in ex vivo animal tissue were compared to those in homogeneous TMPs under spherical-tip micro-indentation technique.

Mechanical measurements were acquired in three types of TMPs, particularly, agar, gelatin, and polyvinyl alcohol (PVA) at distinct concentrations to mimic the acoustic properties of human tissue. Porcine liver and gallbladder tissue, as well as rat liver tissue, were chosen to more closely mimic human tissues and to validate the TMPs' mechanical measurements. Analysis of the generated results in this paper will improve the understanding of the contrast mechanism of imaging techniques that utilize mechanical properties of targets as a primary contrast mechanism (i.e. VA and elastography).

Biological tissues are modeled as viscoelastic materials due to a manifestation of hysteresis on their stress relaxation behavior. The word viscoelastic is a combination of viscous fluidity and elastic solidity, and thus, biological materials under stress and strain exhibit both viscous and elastic behavior. In modeling of materials, such as biological tissues, the linear elastic Hookean spring, which describes elasticity behavior, and the linear viscous Newtonian Dash-pot, which describes viscosity behavior, are used in conjunction to examine and understand the performance of these materials under spring force and displacement.

The linear elastic spring relates stress, defined as the exerted force per unit area, to strain, defined as changes in length with respect to initial length, in a linear fashion by the elastic modulus (E) for solids. During this mechanical process, the material undergoes an instantaneous deformation upon loading and an instantaneous de-straining upon un-loading. The following equation illustrates the simplified relationship in a 1-dimensional (1D) case:

σ=Eε Eq. 8

where σ and ε represent stress and strain in 1D, respectively.

The linear viscous Dash-pot contains a piston-cylinder filled with a viscous fluid and, by definition, it linearly relates stress and strain by the viscosity of the material (η). The following equation represents the linear elastic spring and linear viscous Dash-pot:

σ=η\dot{ε} Eq. 9

Multiple models, such as Maxwell Dash-pot, Kelvin-Voigt, and Hertzian, have been used to characterize the mechanical properties of biological tissues. Of these three models the Hertzian model was chosen to analyze the acquired data because a spherical-tip micro-indenter was used.

The Hertzian viscoelastic contact model relates the exerted force, F, from a rigid sphere with a radius, R, to the elastic modulus, E, and the Poisson ratio, u, of an incompressible material at a given displacement, h. This model evaluates the relaxation behavior of specimens in terms of elastic modulus, Poisson ratio, and indentation depth. Ramp correction factor (RCF) is used in this model to correct the difference between ramp and step loading cycles for each exponential decay.

$$F = \frac{4 E \sqrt{R} {h}^{3 / 2}}{3 ( 1 - {υ}^{2} )} \qquad Eq . 10$$

The Poisson ratio, the ratio of the transverse contracting strain to the elongation strain, of incompressible biological materials is assumed to be between 0.45 to 0.50. However, for sake of simplicity, Poisson ratio was chosen to be 0.50 for all TMPs and ex vivo animal hepatic tissue in this study.

The relaxation response of a step-load (i.e. ideal), rigid, spherical-tip indenter to the material as a function of time and shear modulus, G(t), is illustrated by the following equation:

$${F}_{ideal} ( t ) = \frac{8 \sqrt{R}}{3} {h}_{0}^{3 / 2} G ( t ) \qquad Eq . 11$$

where time-dependent shear relaxation modulus, G(t), is equal to E(t)/3. The observed rise time (t_{R}) in real instances is not an instantaneous step loading process, as opposed to the ideal cases; hence the ramp correction factor (RCF) is used. Therefore, the viscoelastic integral operator for relaxation, where u is a strain function in terms of z, is used and is shown by:

$${F}_{real} ( t ) = {{\int }_{0}}^{t} G ( t - τ ) [ \frac{du ( τ )}{d τ} ] d τ \qquad Eq . 12$$

By combing equations 11 and 12, the real exerted force as a function of time, Fᵣₑₐₗ(t) is:

$${F}_{real} ( t ) = \frac{8 \sqrt{R}}{3} {{\int }_{0}}^{t} G ( t - τ ) [ \frac{d}{d u} {h}^{3 / 2} ( u ) ] du \qquad Eq . 13$$

The Boltzmann integral method is used to solve Eq. 13 and for ramp-loading rate, k, displacement for ramp-hold relaxation can be written as:

h(t)=kt 0≤t≤t_{R} Eq. 14

h(t)=kt_{R}=hₘₐₓ t≥t_{R} Eq. 15

Since the load, F(t), exponentially decays during the process, the solution is expressed as the step-loading relaxation solution adjusted by an RCF due to non-instantaneous ramp loading. Only the first two terms are considered for simplicity in calculations:

F(t)=A₀+A₁ exp(−t/τ₁) Eq. 16

G(t)=B₀+B₁ exp(−t/τ₁) Eq. 17

where τ₁ represents the time constant for the first exponential decay. A₀ and A₁, and B₀ and B₁ represent the fitting constants and the relaxation coefficients, respectively. Only the first term of both the fitting constants and relaxation coefficients (i.e. A₀ and B₀) are computed to compare the material relaxation in terms of applied force with the Wiechert theoretical model. Once all the fitting parameters, A₀ and A₁, have been determined, they are converted to relaxation parameters, B₀ and B₁, using the following equations, where t_{R} is the time that it takes for the force to reach its maximum value:

$$\begin{matrix}{B}_{0} = \frac{{A}_{0}}{{h}_{max}^{3 / 2} ( \frac{8 \sqrt{R}}{3} )} & Eq . 18 \\ {B}_{1} = \frac{{A}_{1}}{( ( {RCF}_{1} ) {h}_{max}^{3 / 2} \frac{8 \sqrt{R}}{3} )} & Eq . 19 \\ {RCF}_{1} = \frac{{T}_{1}}{{t}_{R}} [ exp ( {t}_{R} / {T}_{1} ) - 1 ] & Eq . 20\end{matrix}$$

Instantaneous moduli, E(0), can be computed from the fitted relaxation coefficients using the following equation:

E₀=1.5 G(0)=1.5(B₀+B₁) Eq. 21

The viscosity, η, for each type of material can be calculated as follows:

η=E·τ Eq. 22

The target preparation was divided into two parts: an investigation of 1) TMPs and 2) ex vivo hepatic and bile duct tissues in pre-clinical animal models. For the first part of this study, certain physical geometries and sizes of phantoms were used to satisfy homogeneity and isotropy assumptions for viscoelastic calculations. Additionally, flat, ideal-sized samples of animal tissues were chosen to avoid slippage of the indenter and to reduce any generated noise from the measurements for the second part of the study.

Three TMP types, agar, polyvinyl alcohol (PVA), and gelatin, were fabricated and investigated. These materials were chosen to mimic relevant human anatomical structures (i.e. prostate, oral cavity, liver, and breast) in terms of acoustic and mechanical properties. These water-based gels particularly satisfy the acoustic properties of human tissues, including the speed of sound (about 1540 m/s), attenuation (˜0.5 dB⁻¹ cm⁻¹ MHz⁻¹), and backscatter coefficient (between 10⁻⁵ and 10⁻², between 2 and 7 MHz). These TMPs are also comprised of the primary constituents of tissue, water and protein, and therefore were hypothesized to possess mechanical (i.e. viscoelastic) properties equivalent to that of tissue. Because reported values for the Young's modulus of these types of phantoms fall under the same range as human tissue substituents, these TMPs are ideal candidates to explore the elastic modulus and other types of mechanical properties (i.e. shear modulus, viscosity, and Poisson ratio) of tissue.

These phantoms were also selected due to their distinguished elasticity ranges: PVA falls in the lower end of the elastic moduli spectrum at 1-40 kPa; gelatin is slightly higher at 10-100 kPa, and agar occupies the highest range at 100-200 kPa. Collectively, PVA 14%, 17%, and 20%, agar 2%, 2.5%, and 3%, and gelatin 10%, 15%, and 20%, all percent-by-weight (% wt), were chosen to characterize a spectrum of tissue viscoelasticity, encapsulating both healthy and abnormal tissue states. In particular, 15% gelatin, 3% agar, and 17% PVA were fabricated due to their respective similarities to the acoustic velocity, acoustic impedance, and acoustic attenuation of ideal, healthy human tissue.

For instance, the acoustic velocity in the PVA phantoms were shown to vary from 1520-1540 m/s, which is within the typical range for human soft tissue. Furthermore, PVA phantoms ranging between 14% to 20% are characterized by acoustic impedances similar to those of human breast and skin tissue. Agar TMPs, particularly within the range of 2% to 4%, match the acoustic properties of human prostate tissues. Lastly, the selected gelatin phantom concentrations mimic the acoustic attenuation of human tissues, specifically breast, liver, head and neck, and prostate. The similarity in acoustic properties between these phantoms and human tissues makes this study a crucial stepping stone for imaging modalities like VA. The results will help in establishing the mechanical properties of tissue as the primary agent for optimization of boundary detection and tissue identification. In addition, elastic modulus, viscosity, and relaxation time constant are important parameters for viscoelastic characterization of human tissue, i.e. prostate, oral cavity, liver, and breast and can provide valuable insight for biological modeling.

Agar (Agar, Sigma-Aldrich, St. Louis, Mo.) blocks of varying deionized water/agar concentrations were fabricated in a ˜2×2×2 cm³ plastic mold. Three separate rectangular blocks of agar containing 2, 2.5, and 3% wt of agar were mixed and heated above their gel point (˜90° C.) to maximize cross-linking between the polymers. Increasing the amount of powder in the mixtures is predicted to result in a corresponding increase in the elastic modulus of the phantom.

Gelatin (Porcine Gelatin, Sigma-Aldrich, St. Louis, Mo.) blocks of 10, 15, and 20% wt were used as a second type of TMP. Similar procedures as agar phantoms were used in the preparations of gelatin phantoms; however, the final mixture was placed in a centrifuge at a rotation speed of two rcf (relative centrifugal force) for a period of ˜25 seconds to remove air bubbles from the solution.

Finally, a third type of TMP, PVA (99% hydrolyzed, Sigma-Alrich, St. Louis, Mo.), was fabricated using the same procedure as stated above. Three cubic blocks (same dimensions as agar and gelatin phantoms) containing 14, 17, and 20% wt of PVA were made. Unlike agar and gelatin phantoms, the final PVA mixtures were left at room temperature to cool down for ˜two hours and then placed in a freezer at −20° C. for a period of 24 hours. The phantoms were then taken out of the freezer and allowed to thaw at room temperature for ˜two hours, completing one freezing-thawing cycle. All phantoms, agar, gelatin, and PVA, were made at least 90 minutes prior to measurements to avoid any confounding errors (i.e. dryness and degradation) in the collected data.

Fresh ex vivo liver and bile ducts were harvested from porcine and male Dewey rats shortly after each animal was euthanized under an approved ARC protocol. The samples were stored in saline solution to avoid tissue dryness and degradation to maintain ideal physiological conditions during transportation to the measurement laboratory. Prior to measurements, the tissues were taken out of the saline solution and cut into smaller, ideal pieces measuring ˜15×15×10 mm³ for porcine liver and thinner for the other types of tissues. After dividing the samples into ideal sizes, they were placed in a Petri dish on a balance for viscoelastic measurements.

The isotropic homogeneous TMPs were synthesized in molds with defined geometries. Due to the controlled phantom synthesis, the mean phantom thickness, measuring ˜18 mm, did not vary among each phantom. A total of 10 measurements, 5 for each depth (600 μm and 800 μm), were conducted on each phantom type to calculate the mean and standard error of the mean for each type of measurement. Since the ex vivo hepatic tissues were freshly excised, their thicknesses and geometrical arrangements were not identical to one another. The thicknesses are as follow: ˜11 mm for porcine liver, ˜6 mm for rat liver, and ˜1 mm for porcine gallbladder. These excised samples were relatively flat and of similar lateral dimensions to avoid collection/slippage error. A 2 mm diameter stainless steel sphere was used for the indentation of ex vivo tissues to avoid non-linear behavior. A total of 13 liver samples and two gallbladder samples from two different porcine subjects were examined. Ex vivo indentation measurements consisted of a total of 34 porcine liver indentation measurements, 17 for each depth (300 μm and 400 μm), and 7 measurements of porcine gallbladder tissue at only one depth (100 μm) due to the tissue's width. For rat liver tissues, 14 samples from three different subjects, for a total of 38 liver indentation measurements, 19 for each depth (200 μm and 300 μm), were used to perform this viscoelastic characterization test.

Referring to the schematic diagram illustrated in FIG. 8, a custom system 100 was used to deliver a given strain at a defined displacement for a controlled period of time to probe the absolute instantaneous elastic modulus, viscosity, and time constant of the targets. A 100 nm precision linear stepper motor and controller (LNR50 Series, Thorlabs, Newton, N.J.) were synchronized with a 100 μg precision analytical balance (ML Model, Mettler-Toledo, Columbus, Ohio) to perform the viscoelastic measurements on the targets. The stepper motor was connected to an acrylic rod that displaces a 2 mm diameter stainless steel sphere 102. The sphere 102 was used to create an indentation depth of 300 μm and 400 μm for ex vivo porcine liver samples, 200 μm and 300 μm for ex vivo rat liver, and 100 μm for porcine gallbladder. A slightly larger, 4 mm diameter sphere was used to create indentation depths of 600 μm and 800 μm for all TMP targets 104. Two indentation depths were utilized in order to accurately compute viscoelastic behavior of each target.

For all micro-indentation measurements, the indentation depth was much less than the radius of the sphere to avoid non-linearity and breakage of the target. These indentation distances were chosen based on the thickness of the targets in order to stay within the linear regime, ˜3% strain rate, of the samples. The displacement depth for all targets, except gallbladder tissues, was less than the radius of the indenter in order to avoid any subsequent errors in the recordings.

The indenter displaced downward against the targets, which were placed on an analytical balance pan. This balance served to record the force measurements. The linear motor speed was set as 2 mm/sec. Prior to each measurement, the balance was zeroed to avoid surface tension errors on the acquired data. Shortly after, the target 104 was indented by the sphere with the displacement and speed stated earlier. Once the given displacement was reached, the indenter remained fixed in position, and the target was allowed to relax for approximately 300 sec. During this period, the balance beneath the target recorded the applied force from the target to the sphere. As illustrated in FIG. 8, the sphere 102 induces indentation, smaller than its radius, into the target 104 (Indentation period). After it reaches the maximum depth, it allows the target 104 to relax, with regards to exerted force, (dwell time of relaxation period) in a period of 300 seconds. After completion of the process, the sphere 102 is moved away from the target 104(Retraction period).

a. Results

The viscoelastic behavior of TMPs and ex vivo porcine and rat tissues were examined by a micro-indentation technique using a stainless-steel sphere.

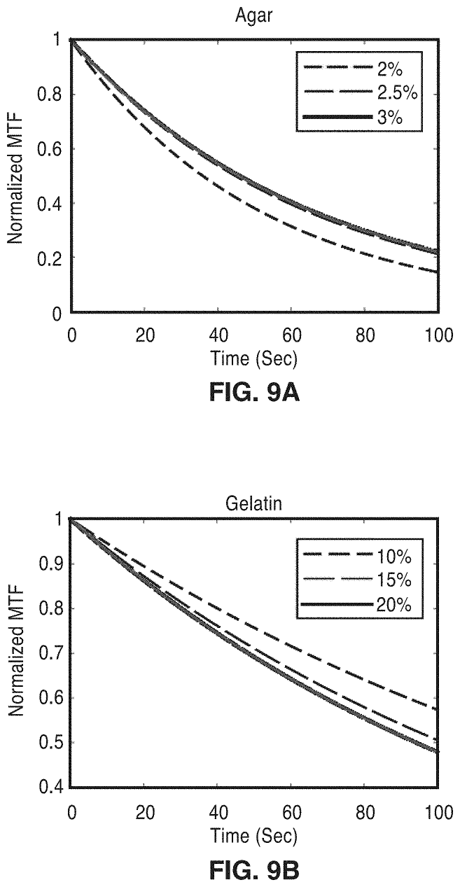

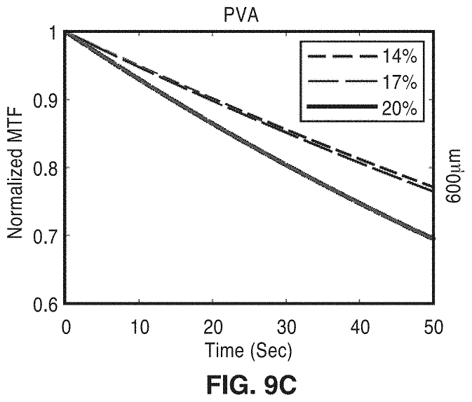

FIG. 9A through FIG. 9C illustrates the relaxation plots for all TMPs and Table 4 shows the instantaneous elastic modulus, viscosity, and time constants (i.e. decay rate) with standard error of the mean for all TMPs at the two different indentation depths. Elastic modulus and viscosity values were calculated by fitting the collected data to a first order exponential decay. From this function, the time constants were calculated using the Hertzian model in MATLAB. PVA phantoms of 14%, 17%, and 20% concentrations possessed elastic moduli of 5.66±0.16 kPa, 9.44±0.20 kPa, and 33.72±1.02 kPa, respectively. The corresponding viscosities were 0.99±0.04 MPa·sec, 1.63±0.08 MPa·sec, and 4.26±0.27 MPa·sec, respectively. Gelatin phantoms of 10%, 15%, and 20% concentrations had elastic moduli of 16.35±0.90 kPa, 45.21±2.24 kPa, and 65.96±3.09 kPa, respectively and the viscosities were 2.77±0.13 MPa·sec, 6.37±0.31 MPa·sec, and 8.80±0.45 MPa·sec, consistently. Similarly, for agar phantoms of 2%, 2.5%, and 3% concentrations, the mean calculated elastic moduli were 104.64±3.91 kPa, 135.48±4.65 kPa, and 195.17±4.28 kPa, and the viscosities were 5.80±0.21 MPa·sec, 9.58±0.61 MPa·sec, and 14.24±0.84 MPa·sec, respectively. The relatively small standard error of the mean for elastic modulus and viscosity measurements of each phantom type shows a very small deviation from the mean in both indentation depths for all phantoms.

As shown by the calculations in Table 4 and FIG. 9A through FIG. 9C, as the concentration of gelatin and PVA TMPs increases, the calculated time constant decreases. In the case of agar TMPs, the exact opposite was observed: increases in the concentration of the TMP is associated with an increase in the time constant.

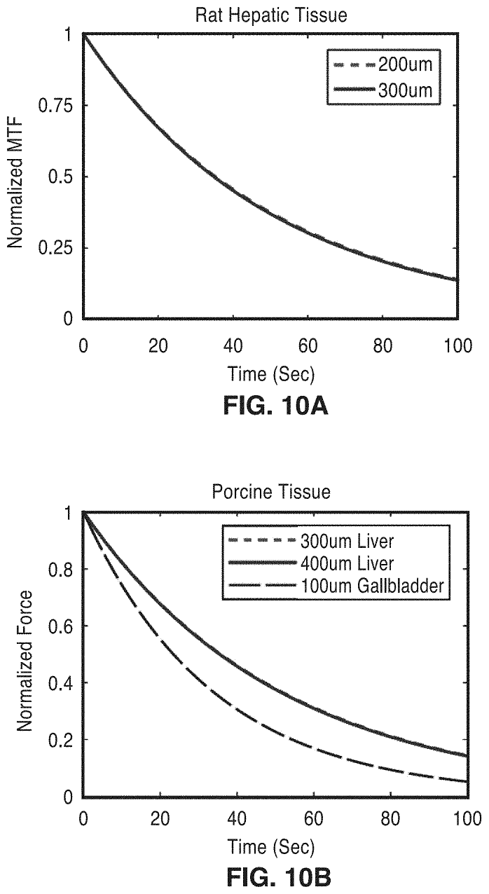

FIG. 10A and FIG. 10B illustrates the relaxation plots and Table 5 shows the instantaneous elastic modulus, viscosity, and time constant for ex vivo porcine liver, porcine gallbladder, and rat liver. For porcine tissues, the mean elastic moduli for liver and gallbladder were 2.55 kPa and 4.73 kPa, respectively. The mean viscosity values of liver and gallbladder tissue were 0.14 MPa·sec. and 0.18 MPa·sec, respectively. With regards to rat liver, the mean elastic modulus was 2.76 kPa and the viscosity was 0.15 MPa·sec. All ex vivo tissues had small variations, as shown by their respective standard errors of the mean in Table 5.

During gallbladder tissue preparation, excess bile was removed from the tissue to avoid slippage of the sphere indenter, which could have potentially affected the viscoelastic measurements. The standard error of the mean for both rat and porcine liver were very small due to consistency of the data; however, that error value was a bit higher for the gallbladder. This could be due to the remnants of bile that could not be removed, and the small tissue thickness of the gallbladder samples.

Moreover, the time constants and relaxation curves for both rat liver and porcine liver were very similar. Both liver specimens also had similar elastic modulus and viscosity values, further suggesting that the viscoelasticity is similar for these two types of tissue. In comparison to the liver tissues, the porcine gallbladder tissues demonstrate a smaller time constant, though a higher elastic modulus and viscosity, illustrating the unique viscoelastic characteristics of gallbladder tissue.

This section evaluates the characterization of the viscoelastic properties of TMPs and ex vivo animal tissues by examining elastic modulus, viscosity, and relaxation time constant in a unique fashion. This study utilizes the Hertzian mathematical model and explores spherical-tip micro-indentation to analyze the observed relaxation curves. The generated relaxation curves are fitted to a first order exponential fit with a relatively high R² value (i.e. >0.9) for all cases. Other models, such as Maxwell Dash-pot and Kelvin-Voigt, were also considered for the evaluation of the collected data; however, because spherical-tip micro-indentation was used, the Hertzian mathematical model was best suited for the analysis.

Our results show that for mechanical modeling of biological tissue with viscoelastic behaviors, both elasticity and viscosity should be considered. The TMP elasticity measurements show three unique elasticity ranges: PVA: 1-40 kPa (low), gelatin: 10-100 kPa (medium), and agar: 100-200 kPa (high). Along with their corresponding viscosity values, the TMPs were examined to cover a wide range of viscoelasticity for soft tissues. Ex vivo liver and gallbladder tissues of porcine and rat were also explored to compare their viscoelastic measurements with TMPs. This work originated from our previous work that correlated elastic properties of TMPs with radiation force intensity measurements from a previously described vibroacoustography system. The results obtained from that work encouraged the examination of other mechanical properties (i.e. viscosity and time constant) to develop a mathematical model to explore the observed behavior of targets under the acoustic radiation force of ultrasound imaging modalities (i.e. vibroacoustography and elastography).

Due to our unique experimental set-up and measurement calculations, direct comparisons are not forthright for each aspect of the study; however, similar approaches have been made. The overall trend demonstrated in our study showed higher concentrations elucidate higher elastic modulus values.

Viscosity and time constant characterization were other features that were analyzed. To the best of our knowledge, there are no straightforward comparisons with the literature, but there are related works on shear viscosity of soft tissues and solids characterizations.

The calculated time constants for the gelatin and PVA TMPs followed the same trend: steady decrease as concentration increased. Although, this trend switched in the case of the agar phantoms. This result may be due to the structural properties as well as the cross-linking of the agar substituents within the phantom.

The second half of this study was devoted to the investigation of ex vivo porcine and rat tissues. Porcine liver and gallbladder tissues, along with rat livers, were tested using the same technique that was used for TMPs. The tissues were excised fresh, immediately after the animals were euthanized, and kept in a saline solution during transport to maintain tissue integrity. Many scientists use pre-conditioning technique with cyclic deformations for their ex vivo sample preparations. However, this technique was not used in this study because the generated results were already in steady-state mode and the elastic modulus, viscosity, and time constant calculations were consistent with very low standard error of the mean. Moreover, the duration of examination for each sample was relatively short, ˜15 minutes per sample, demonstrating the perseverance of the physiological conditions and mechanical feasibility throughout the span of the measurement.

Similar to the TMP viscoelasticity results, there is no direct comparison for our generated ex vivo porcine and rat tissue viscoelasticity data within the literature, but some methodologies have been developed for this comparison. Elastic modulus and viscosity were not directly calculated in this study, but the calculated time constant can alone be a precursor to the viscoelastic measurements since in theory, the time constant is the product of elastic modulus, analogous to Young's modulus, and viscosity, as seen in Eq. 22.

Typically, healthy in vivo human parenchyma liver has shear modulus and viscosity of μ₁=2.06±0.26 kPa and μ₂=1.72±0.15 Pa·sec, calculated using Magnetic resonance elastography (MRE). Healthy in vivo porcine liver values, obtained from 9 different measurements of Shearwave Dispersion Ultrasound Vibrometry (SDUV), are p=2.2±0.63 kPa and μ₂=1.96±0.34 Pa·sec. Lastly, normal in vivo parenchyma rat liver values are μ₁=1.76±0.37 kPa and μ₂=0.51±0.04 Pa·sec, again by using MRE. By assuming liver to be an incompressible material, the Poisson ratio can be estimated to be 0.50, and the elastic modulus can be calculated using its relationship with shear modulus through the following equation:

$$μ = \frac{E}{2 * ( 1 + υ )} \qquad Eq . 23$$

Using Eq. 23, the approximate elastic modulus for hepatic tissue is calculated to be ˜6.16 kPa for humans, ˜6.58 kPa for porcine, and ˜5.26 kPa for rats. However, both our calculated elastic modulus and viscosity values for porcine and rat livers are smaller than the values reported literature. One reason for this discrepancy could be due to the assumptions (i.e. tissue homogeneity, density, the implemented model (Voigt model)), as well as the detection scheme they used for calculating these values. Since micro-indentation was used in this study, some of these assumptions, particularly density and homogeneity, can be ignored, even though liver tissues are not homogeneous. Moreover, placing a tissue on a hard surface induces external forces (i.e. stress) that are not present in the in vivo state. Other factors, such as capsules, blood flow, cellular association, amongst other in vivo conditions, need to be taken into account for accurate measurements. Fortunately, our measurements, for both rat and porcine liver, were free of these potentially confounding in vivo factors. However, the measured viscoelastic properties were different when comparing the ex vivo values to those of TMP data. This dissimilarity can be explained by the physiological conditions discussed earlier.

Porcine gallbladder was the third tissue type characterized in this study. We sought to use the viscoelastic properties of the gallbladder to differentiate it among other close organs, particularly the porcine liver. As presented in Table 5, the elastic modulus was ˜2 kPa higher than the liver and there was a difference of ˜20 seconds in the time constants. This illustrates that there exists a distinct difference in viscoelastic properties between the two porcine tissue types, liver and gallbladder. Even though the two organs are relatively close to one-another, their respective viscoelastic properties can be used to differentiate the two, highlighting clear boundary distinction between the two tissue types. Moreover, imaging modalities (i.e. vibroacoustography and elastography) can utilize these mechanical properties of tissue as a contrast mechanism in order to recognize and distinguish different tissue types within a particular area of interest. The clear, distinguished viscoelastic properties of tissue can lead way to these imaging modalities, particularly vibroacoustography, as ideal, non-invasive approaches for tissue identification and characterization in the field of medicine.

Furthermore, an embodiment of this technology uses spherical-tip micro-indentation technique along with Hertzian model to analyze and characterize the generated relaxation data, with a relatively controlled protocol. Therefore, complete mechanical characterization, particularly those which assess stress and strain rates that bring the tissue to failure, in both in vivo and ex vivo cases, are critical for establishing mathematical models. These mathematical models can then be used in conjunction with imaging modalities (i.e. vibroacoustography) to accurately and precisely describe viscoelastic mechanical properties of targets for identification and characterization.

This study focuses on characterization of the viscoelastic properties of TMPs and ex vivo animal tissues. TMPs can be synthesized to mimic the acoustic and mechanical properties of biological tissues, but accurate characterization and modeling still requires fresh biological tissues. This study also investigated the mechanical behaviors of ex vivo porcine and rat tissues; however, direct characterization and evaluation with ex vivo and possible in vivo biological organs is still a necessity for further validation of mechanical properties. The conducted experiments in this study may bolster the possibility of using tissue mechanical properties, particularly viscoelasticity, as the primary contrast mechanism for developing new imaging modalities, like vibroacoustography and elastography. To this end, insights gained from assessment of animal tissues have helped researchers better understand the underlying mechanical behavior of biological tissue. However, an improved ex vivo experimental setting (i.e. closer replication to in vivo physiological parameters) may be implemented for optimal viscoelastic measurements. Nevertheless, characterization and evaluation of ex vivo animal hepatic and gallbladder tissues under micro-indentation techniques, among other mechanical techniques, provide additional information that can guide researchers and scientists in modeling and investigative approaches for tissue response under a static force. Given that biological tissues behave as viscoelastic materials, both viscosity and elasticity should be considered when examining them. In the medical field, palpation, the gold standard for detecting abnormalities, only utilizes the elasticity to characterize tumor boundary regions, which is perhaps the reason for the lack of quality of tissue differentiation in real-time procedures. Additionally, soft target characterization may play a salient role in target imaging (i.e. vibroacoustography and elastography techniques), material characterization, and non-destructive testing (NDT).

4. Mathematical Modeling of the Contrast Mechanism

Biological tissues are modeled as viscoelastic materials due to a manifestation of hysteresis on relaxation of the stress in stress vs. strain curves. Gelatin, agar, and PVA and other ex vivo pre-clinical models such as liver, gallbladder, and HNSCC specimens are appropriate representations of tissue substituents that were used in this work to establish the viscoelastic mechanical properties of targets as the dominant contrast mechanism for VA imagery. In the previous sections, the Hertzian model and contact mechanics model were used to examine the viscosity, elastic moduli, and time constant of these materials under applied force and displacement. The results were then compared to VA signal intensity to generate a relationship between the two techniques. An inverse relationship was demonstrated between VA signal intensity and elastic moduli of TMPs using contact mechanics, but a mathematical model that directly relates VA generated signals to mechanical properties has not been established. The purpose of this chapter is to develop a mathematical relationship based on target's geometry, mechanical properties, and acoustic properties to further investigate and verify the contrast mechanism of the VA imagery.

The word viscoelastic is a combination of viscous fluidity and elastic solidity and thus materials under stress and/or strain exhibit both viscous and elastic behavior. Moreover, in modeling materials such as biological tissues, linear elastic Hookean spring, elasticity behavior, and linear viscous Newtonian Dash-pot, viscosity behavior, are used to examine and understand the performance of these materials under spring force and displacement. Various simple mechanical models such as Maxwell and Kelvin-Voigt models are used to describe this behavior.

The scenario of time dependence of viscoelastic response is similar to the time dependence of electrical circuits, and thus ordinary differential equations can be utilized to examine their characteristics. In Maxwell's model, Hookean spring and a Newtonian Dash-pot are connected in series and so the stress on each element is the same and equal to the initial stress and the strain is the addition of each element.

$$\begin{matrix}σ = {σ}_{s} = {σ}_{d} & Eq . 24 \\ ɛ = {ɛ}_{s} + {ɛ}_{d} = \frac{{σ}_{s}}{E} + \frac{{σ}_{d}}{η} & Eq . 25\end{matrix}$$

where σₛ and εₛ represent stress and strain of Hookean spring and σ_{d} and ε_{d} represent stress and strain of Newtonian Dash-pot, respectively. At equilibrium, the stress of each element will be the same, and by multiplying by E and substituting time constant, τ=η/E, the total strain in Maxwell model becomes:

$$E ɛ = σ ( 1 + \frac{1}{τ} ) \qquad Eq . 26$$

One way to test the viscoelastic behavior of the target under Maxwell model is stress relaxation. In this test, the material initially will undergo constant strain, ε0, equating to the initial condition of σ₀=Eε₀. So, by integrating using variable separation, the total stress that was illustrated in the Maxwell model becomes:

$$σ ( t ) = {σ}_{0} exp \frac{- t}{τ} \qquad Eq . 27$$

The stress relaxation behavior is denoted by an exponential decay with time constant, τ, which shows as the applied strain on the target decreases, the measured stress decreases in an exponential decay fashion. This shows the non-linear behavior of viscoelastic materials when under strain using Maxwell model.

In Kelvin-Voigt's model, Hookean spring and a Newtonian dash-pot are connected in parallel and so the strain on each element is the same and equal to the initial strain, while the stress is the addition of the stress on each element. For this model, it is assumed that the elements will not bend in response to a force or displacement, so the strain on each element remains equal:

ε=εₛ=ε_{d} Eq. 28

σ=σₛ+σ_{d}=Eε+ηε=ε(E+η) Eq. 29

One way to test the viscoelastic behavior of the targets under Kelvin-Voigt model is by creep recovery test. In this test, the material is subjected to a constant stress, σ₀. After applying the constant stress, the spring will want to stretch, but it is held back by the Dash-pot due to its delayed reaction, so the stress is initially used by the Dash-pot and as a result the creep curve starts with an initial slope of σ₀/η. However, after a defined time period, the spring takes all the stress that was inputted and sets up an initial condition of ε₀=σ₀/E illustrating the deformation of a pure elastic element. Therefore, the total strain in the system using Kelvin-Voigt model:

$$ɛ ( t ) = {ɛ}_{0} ( 1 - exp \frac{- t}{τ} ) \qquad Eq . 30$$

In this case, the system will undergo an initial stress causing an initial deformation, strain, in a period of τ, retardation time, and then the stress is removed and the creep-recovery, strain, will be collected in an exponential decay fashion. The retardation time, τ, is the same as the time constant in the Maxwell model. The shorter the retardation time, the faster the creep straining.

Moreover, when the load is removed from the target, the spring reacts instantaneously, but the Dash-pot cannot recover, and as a result there appears an elastic recovery.

As mentioned before, the resultant dynamic radiation force, Fr(Δω) from an acoustic vibration field on a homogeneous isotropic sphere immersed in a homogeneous fluid, i.e. propagating medium, is given by:

Fr(Δω)=Sdᵣ [symbol] E [symbol] Eq. 31

where S=πr² is the vibrated area of the target with radius of r, [symbol] E [symbol] =p(t)²/ρ₀c² is the time averaged energy density of the ultrasonic waves with [symbol] p(t)=2P₀[1+cos(Δω)t+Δσ] for the vibroacoustography technique, and dr is the radiation force function that depends on the material properties of the target and the medium, usually expressed as a function of k, where k is the wave number of the incident wave, 2π/λ, and r is the target's radius.

The velocity of an oscillating sphere in a viscoelastic medium using Oestreicher can be calculated as:

$$V = \frac{{F}_{r}}{{Z}_{r} + {Z}_{m}} \qquad Eq . 32$$

where Zᵣ and Zₘ are radiation and mechanical impedances, respectively. Zᵣ can be defined as:

$$\begin{matrix}{Z}_{r} = - j \frac{4 π {r}^{3}}{3} ρΔω ( \frac{( 1 - \frac{3 j}{rh} - \frac{3}{{r}^{2} {h}^{2}} ) - 2 ( \frac{j}{rh} + \frac{1}{{r}^{2} {h}^{2}} ) ( 3 - \frac{{r}^{2} {k}^{2}}{jrk + 1}}{( \frac{j}{rh} + \frac{1}{{r}^{2} {h}^{2}} ) \frac{{r}^{2} {k}^{2}}{jrk + 1} + ( 2 - \frac{{r}^{2} {k}^{2}}{jrk + 1} )} ) & Eq . 33 \\ k = \sqrt{\frac{{ρΔω}^{2}}{( 2 μ + λ )}} and h = \sqrt{\frac{{ρΔω}^{2}}{μ}} & Eq . 34\end{matrix}$$

where μ=μ₁+jΔωμ₂ and λ=λ₁+jΔωλ₂ are Lame's constants, ρ is the density of the medium, c is the speed of sound of the medium, μ₁ and μ₂ are the shear elasticity and viscosity of the medium, respectively, and λ₁ and λ₂ are the bulk elasticity and viscosity of the medium, respectively. Using Newton's second law, the resultant force on an object with mass m and measured velocity v is defined as:

$$F = m \frac{d}{dt} {ve}^{j {Δ}_{ω t}} = jm Δ ω {ve}^{j {Δ}_{ω t}} \qquad Eq . 35$$

The mechanical impedance is defined as the ratio of the applied force to the resulting velocity:

$${Z}_{m} = \frac{- F}{{ve}^{j Δ ω t}} = - jm Δω = - j \frac{4 π}{3} {r}^{3} ρ s Δω \qquad Eq . 36$$

where the mechanical impedance is in terms of the object's volume,

$$\frac{4 π}{3} {r}^{3} ,$$

beat frequency, Δω, and object's density, ρₛ.

Using phasor notation for the calculated velocity, the velocity becomes:

$$V = \frac{\vert {F}_{r} \vert {∠ϕ}_{r}}{\vert {Z}_{r} \vert {∠ϕ}_{r} + \vert {Z}_{m} \vert {∠ϕ}_{m}} = \vert \frac{{F}_{r}}{Z} \vert {∠ϕ}_{r} - {∠ϕ}_{z} \qquad Eq . 37$$

Based on the Eqs. 33, 32, 36 and 37, we can calculate the shear velocity by the assumption of continuity, homogeneity, and isotropy of the medium for homogeneous isotropic targets of varying density over a range of beat frequencies.

5. Mathematical Model Formulation

As discussed in chapter one, the radiation force generated from the confocal transducer in VA technique is confined to a focus plane to image a target of interest. It is only at the focus spot that the beat frequency is generated from the intersection of two CW ultrasonic waves. As a result of this generation, the object will vibrate at the beat frequency, Aw, and start emitting acoustic radiation that is a function of its geometry, surrounding medium, and mechanical and acoustic properties. The relationship between the emitted acoustic radiation pressure and the object is as follows:

P_{(Δω)}(\vec{r})=ρ₀c²H_{(Δω)}(l)Q_{(Δω)}(\vec{r})F_{r(Δω)} Eq. 38

where the detected pressure, P_{Δω} is in terms of medium density, ρ₀, speed of sound in the medium, c, medium transfer function, H_{(Δω)}(l), the radiation force, F_{(Δω)}, and Q_{(Δω)}(\vec{r}), total acoustic outflow by an object per unit force which itself is in terms of the ultrasound pressure, P, and the ultrasound characteristics of the object, Y, and (\vec{r}), a radial vector on the focal plane The function QΔω( [symbol] ) includes target's vibrating area, geometry, and its mechanical impedance. The mechanical impedance itself has two components: one arising from inertia, i.e. geometry and mass, m, friction, and viscoelasticity, i.e. elastic moduli, E, and viscosity, η:

$${Q}_{( Δω )} ( \vec{r} ) = \frac{A}{( Zm + Zr )} \qquad Eq . 39$$

Eq. 39 demonstrates that the generated acoustic radiation pressure is dependent on both mechanical and radiation impedance of the target as well as the area of the target, A. In the case of radiation impedance, the target is assumed to be a rigid piston with radius r set in a plane wall. Therefore, the radiation impedance will become:

$$\begin{matrix}{Z}_{r} = π {r}^{2} ( {R}_{r} - {jX}_{r} ) \\ where {R}_{r} = ρ c [ 1 - [ 1 - \frac{c}{Δω r} {J}_{1} ( \frac{c}{2 Δω r} ) ] and & Eq . 40 \\ {X}_{r} = \frac{4 ρ c}{π} {{\int }^{\frac{π}{2}}}_{0} sin [ \frac{2 Δω r}{c} cos α ] {sin}^{2} ( α ) d α . & Eq . 41\end{matrix}$$

The real part of the radiation impedance, Rᵣ, is in terms of the beat frequency, Δω, medium acoustic properties, ρc, and J₁(a), the first order Bessel function of the first kind. The imaginary part, Xᵣ, is in terms of α, which represents the angle between the detector and the center of the target. Both parts in the radiation impedance are in terms of

$$( \frac{r Δω}{c} )$$

and in the case of small targets:

$${A}_{s} \frac{2 Δω a}{c} < 0.5 \to {R}_{r} = \frac{2 {Δω}^{2} {a}^{2}}{2 {c}^{2}} and as \frac{2 Δω a}{c} < 1 {X}_{r} = \frac{8 ω a}{3 π c} \qquad Eq . 42$$

Therefore, the radiation frequency term becomes a constant for small targets.

In the case of mechanical impedance, Zm, the general solution becomes:

Zₘ=Rₘ−jXₘ=Rₘ−j(mΔω−k/Δω) Eq. 43

where Rₘ represents the mechanical resistance analogous to electrical resistance and damper (viscosity in Dash-pot), m represents the mass analogous to electrical inductance and mass of the target, k represents the stiffness analogous to capacitor and spring constant (elasticity), and Δ_{ω} represents the beat frequency at the focus plane.

The last term in Eq. 38 is H_{(Δω)}(l), the medium transfer function, which is in terms of the target's radius, observation point, acoustic properties of the medium, and boundary conditions based on Morse derivation is:

$${H}_{( Δω )} ( l ) = j \frac{{Δω}^{2}}{{c}^{2}} [ \frac{exp ( \frac{j Δ ω 1}{c} )}{4 π 1} ( \frac{2 {J}_{1} ( \frac{Δω r}{c} )}{\frac{Δω r}{c} sin ϑ} \times \frac{cos ϑ}{cos ϑ + \frac{{ρ}_{0} c}{{Z}_{B}}} ) ] \qquad Eq . 44$$

where l is the distance from the observation point, detector, to the center of the target, ϑ, is the angle between the observation and the imaging axis, of the curved transducer, and Z_{B} is the acoustic impedance of the boundary, the ratio between the pressure and the normal fluid velocity at a point on the target.

As discussed in this work, the generated acoustic pressure depends on the medium transfer function, H_{(Δω)}(l), radiation force, Fr(Δω), and the acoustic outflow, Q_{Δω}( [symbol] ); however, the only parameter that includes the mechanical properties of target, i.e. elasticity and viscosity, is the imaginary part of the mechanical impedance of the acoustic outflow parameter. The final expression for the generated acoustic pressure is as follows:

$${P}_{Δω} = j \frac{{Δω}^{2}}{{c}^{2}} {d}_{r} {p ( t )}^{2} S [ \frac{exp ( \frac{j Δ ω 1}{c} )}{4 π 1} ( \frac{2 {J}_{1} ( \frac{Δω r}{c} sin ϑ )}{\frac{Δω r}{c} sin ϑ} \times \frac{cos ϑ}{cos ϑ + \frac{{ρ}_{0} c}{{Z}_{B}}} ) ] \frac{A}{( {Z}_{m} + {Z}_{r} )} \qquad Eq . 45$$

where p(t)² is the amplitude of the incoming ultrasonic waves, dᵣ is the radiation force function, S is the vibrated area of the target, and A is the surface area of the vibrated target, which in a case of small targets, i.e. ( )→small, they are assumed to be the same. Moreover, the generated acoustic pressure in Eq. 45 is inversely proportional to Zₘ, the only factor that includes the stiffness, k, of the target, and further confirms our experimental results. However, to directly evaluate the effect of viscoelasticity of the target as a function of the beat frequency, the acoustic outflow needs to be examined. So, the behavior of targets with different densities, illustrating different tissue constituents, were simulated to determine the resonant beat frequency for each constituent.

Algorithm 1, provided below, shows exemplary instructions in the form of Matlab code that may be implemented as application software 74 (FIG. 3) for practicing the methods of the present description.

Algorithm 1: Matlab Code for Acoustic Outflow of Biological Tissues

| | |
|---|---|
| %%%%% Variables %%%%% | |
| w = linspace((1000),(4000),(1000)); | %difference frequency (Hz) |
| c = 1500; | %speed of sound in water |
| a = 0.0005; | %radius of sphere (m) |
| A = pi*a\hat{ }2; | %area of sphere (m\hat{ }2) |
| V = (4*pi*a\hat{ }3)/3; | %volume of sphere (m\hat{ }3) |
| Rho = linspace(800,1200,1000); | %density (kg/m\hat{ }3) |
| Rho_water = 1000; | |
| Rho_fat = 950; | |
| Rho_soft = 1043; | |
| Rho_muscle = 1050; | |
| m = Rho*V; | |
| m_water = Rho_water*V; | |
| m_fat = Rho_fat*V; | |
| m_soft = Rho_soft*V; | |
| m_muscle = Rho_muscle*V; | |
| k_water = linspace(1,10,1000); | %Elastic moduli (kPa) |

k_fat = linspace(1,20,1000);
k_soft = linspace(20,50,1000);
k_muscle = linspace(50,100,1000);
R_r_water = Rho_water.*A*c*(w.\hat{ }2*a\hat{ }2)./(2*c\hat{ }2); %real radiation
impedance
X_r_water = Rho_water.*A*c*(8*w*a)./(3*pi*c); %imaginary radiation
impedance
Z_r_water = sqrt((R_r_water).\hat{ }2+(X_r_water).\hat{ }2);
R_m = 0.00000001*linspace(100,500,1000);
X_m_water = (m_water.*w)−(k_water./w);
X_m_fat = (m_fat.*w)−(k_fat./w);
X_m_soft = (m_soft.*w)−(k_soft./w);
X_m_muscle = (m_muscle.*w)−(k_muscle./w);
Z_n_water = sqrt((R_m).\hat{ }2 + (X_m_water).\hat{ }2); %Mechanical Impedance of
small object
Z_n_fat = sqrt((R_m).\hat{ }2 + (X_m_fat).\hat{ }2); %Mechanical Impedance of fat
Z_n_soft = sqrt((R_m).\hat{ }2 + (X_m_soft).\hat{ }2); %Mechanical Impedance of soft
Z_n_muscle = sqrt((R_m).\hat{ }2 + (X_m_muscle).\hat{ }2); %Mechanical
Impedance of muscle
Q_water = A./(Z_r_water+Z_m_water); %Acoustic energy output of small
object
Q_fat = A./(Z_r_water+Z_m_fat); %Acoustic energy output of fat
Q_soft = A./(Z_r_water+Z_m_soft); %Acoustic energy output of soft tissue
Q_muscle = A./(Z_r_water+Z_m_muscle); %Acoustic energy output of
muscle
Q_norm_water = Q_water./max(Q_water);
Q_norm_fat = Q_fat./max(Q_fat);
Q_norm_soft = Q_soft./max(Q_soft);
Q_norm_muscle = Q_muscle./max(Q_muscle);
figure; hold on;
plot(log(w/1000), Q_norm_fat,’r’,’linewidth’,2)
plot(log(w/1000), Q_norm_soft,’b’,’linewidth’,2)
plot(log(w/1000),Q_norm_muscle,’k’,’linewidth’,2)
hold off;

a. Results

For this simulation, human adipose tissue, soft tissue, and muscle in spherical shapes were chosen to mimic actual scenarios when imaging human tissue. The acoustic outflow of each type was compared as a function of their generated resonant frequency peak. The three tissues had water with ρ=1000 kg/m³ and c=1500 m/s as their propagating media for the ultrasonic waves. The spherical geometry was chosen to ensure isotropic homogeneity for simplification of calculations. In terms of radiation impedance, Zᵣ, it was assumed to be a constant since the spherical targets, r=500 μm, were much smaller than the wavelength of the beat frequency range, 1-100 kHz. For these three tissue types, the real part of the acoustic outflow, Rm, was assumed to be zero for all types. For the imaginary part, adipose tissue was assumed to have ρ=950 kg/m³ and k=1-20 kPa, soft tissue ρ=1043 kg/m³ and k=20-50 kPa, and muscle ρ=1050 kg/m³ and k=50-100 kPa, respectively. These values were based on given density and elastic modulus.

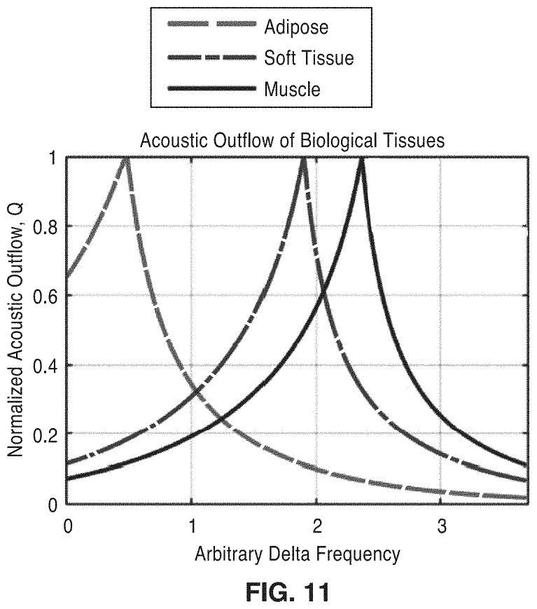

FIG. 11 represents the generated results from this simulation, showing a plot of acoustic outflow of biological tissues, specifically adipose, soft tissue, and muscle, as a function of resonant beat frequency. As the targets become stiffer, i.e. increase in density as well as elastic modulus, the peak resonant frequency for each tissue type increases. This positive correlation was shown above for TMPs, and is now confirmed by the results of this simulation. Moreover, this allows for the characterization of tissue stiffness and creation of databases for tissue vibroacoustic responses based on their mechanical properties.

The simulation results detailed above verify the hypothesis that the tissue viscoelastic properties are the primary contrast mechanism in the generation of VA signal in the vibroacoustography imaging system. A positive correlation between mechanical properties of tissues and the beat frequency was observed. The simulation results in various human tissue constituents illustrated here as well as tissue mimicking phantom results described above show a good proof of concept. This may further help with VA resonant frequency characterization for different tissue types for the creation of databases for additional VA technique investigation.

6. Transient Analysis of Vibroacoustography Signals

In the current iteration of vibroacoustography imaging, ultrasound waves of two different frequencies are focused to a single voxel, and the mixing of those two frequencies results in a beat frequency signal of lower frequency (df), which is then detected by a hydrophone. The hydrophone is fed into a lock-in amplifier to isolate the lower frequency signal and output a single magnitude and phase value for that voxel. The signal received and recorded is a steady-state response of the tissue vibrating at the lower frequency, df. The magnitude/phase values correlate to the viscoelastic mechanical properties of the tissue in a mathematical model as described earlier. To obtain 2D images of the tissue of interest, the system is scanned mechanically point by point, recording the magnitude and phase at each point, until the region is fully imaged.

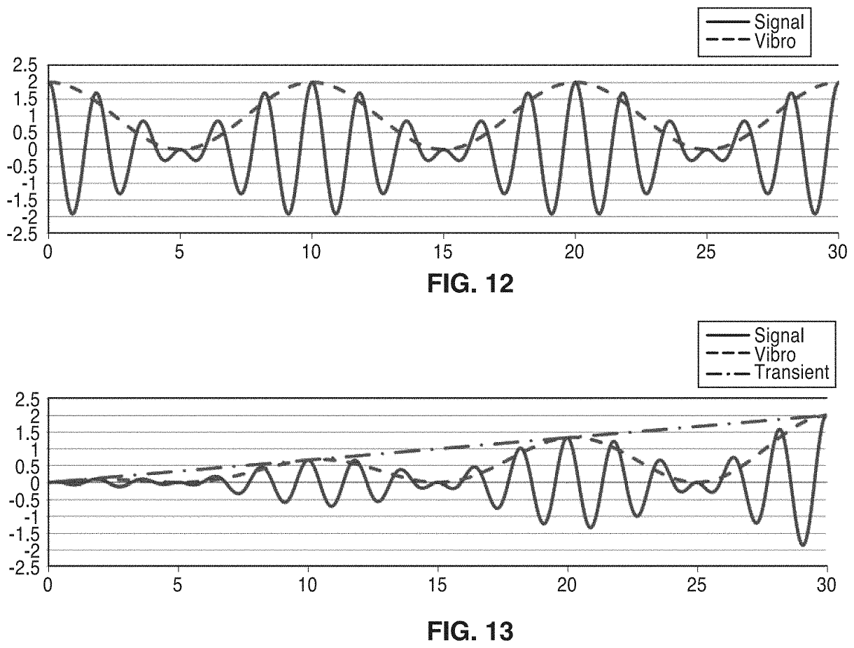

As stated, the df magnitude/phase values are recorded when the tissue is vibrating at a steady-state, so the amplitude of the differential frequency is consistent when recorded by the lock-in amplifier (FIG. 12). There is information, however, in the tissue's initial response to the excitation frequencies, as it transitions from rest-state to vibrating at steady-state. This is the transient analysis of vibracoustography signal, and the mechanical properties of the tissue of interest will determine how quickly the tissue responds to the initial vibroacoustic excitation waves (FIG. 13).

The tissue's transient response can either be a linear growth to steady-state, an exponential growth to steady-state, or a combination of the two. The key to capturing this data is the speed of data acquisition of the signal from the hydrophone. If acquiring data directly from the hydrophone, the sampling rate will need to be at least twice the sum of the two excitation frequencies as to capture the carrier wave of the differential frequency signal. Then the signal will be rectified and filtered to isolate the vibro frequency, df. If data is taken from the lock-in amplifier, two factors come into play to accurately measure the transient response. First, the acquisition rate must be twice the vibro frequency, df. Second, the time constant of the lock-in amp must be at least half of the period of the vibro frequency, df. This gives the lock-in amp adequate time to correctly isolate the vibro frequency, df, and still allow for enough sampling time to capture the rise of vibroacoustic signal.

The same transient analysis of vibroacoustic signal for initial excitation can also be applied to the relaxation process of the tissue after the excitation ultrasound waves are stopped. It measures the tissues viscoelastic response to inertial changes, which correlates to its mechanical properties.

Just as a frame of reference, this transient analysis is similar in nature to the optical analysis of fluorescence. Imaging can capture the absolute fluorescence amount (steady-state), or the fluorescence-lifetime values (transient response) of different fluorophores. Each type of analysis gives similar information about the sample of interest; one is just more specific than the other.

Embodiments of the present technology may be described herein with reference to flowchart illustrations of methods and systems according to embodiments of the technology, and/or procedures, algorithms, steps, operations, formulae, or other computational depictions, which may also be implemented as computer program products. In this regard, each block or step of a flowchart, and combinations of blocks (and/or steps) in a flowchart, as well as any procedure, algorithm, step, operation, formula, or computational depiction can be implemented by various means, such as hardware, firmware, and/or software including one or more computer program instructions embodied in computer-readable program code. As will be appreciated, any such computer program instructions may be executed by one or more computer processors, including without limitation a general-purpose computer or special purpose computer, or other programmable processing apparatus to produce a machine, such that the computer program instructions which execute on the computer processor(s) or other programmable processing apparatus create means for implementing the function(s) specified.

Accordingly, blocks of the flowcharts, and procedures, algorithms, steps, operations, formulae, or computational depictions described herein support combinations of means for performing the specified function(s), combinations of steps for performing the specified function(s), and computer program instructions, such as embodied in computer-readable program code logic means, for performing the specified function(s). It will also be understood that each block of the flowchart illustrations, as well as any procedures, algorithms, steps, operations, formulae, or computational depictions and combinations thereof described herein, can be implemented by special purpose hardware-based computer systems which perform the specified function(s) or step(s), or combinations of special purpose hardware and computer-readable program code.

Furthermore, these computer program instructions, such as embodied in computer-readable program code, may also be stored in one or more computer-readable memory or memory devices that can direct a computer processor or other programmable processing apparatus to function in a particular manner, such that the instructions stored in the computer-readable memory or memory devices produce an article of manufacture including instruction means which implement the function specified in the block(s) of the flowchart(s). The computer program instructions may also be executed by a computer processor or other programmable processing apparatus to cause a series of operational steps to be performed on the computer processor or other programmable processing apparatus to produce a computer-implemented process such that the instructions which execute on the computer processor or other programmable processing apparatus provide steps for implementing the functions specified in the block(s) of the flowchart(s), procedure (s) algorithm(s), step(s), operation(s), formula(e), or computational depiction(s).

It will further be appreciated that the terms “programming” or “program executable” as used herein refer to one or more instructions that can be executed by one or more computer processors to perform one or more functions as described herein. The instructions can be embodied in software, in firmware, or in a combination of software and firmware. The instructions can be stored local to the device in non-transitory media, or can be stored remotely such as on a server, or all or a portion of the instructions can be stored locally and remotely. Instructions stored remotely can be downloaded (pushed) to the device by user initiation, or automatically based on one or more factors.

It will further be appreciated that as used herein, that the terms processor, hardware processor, computer processor, central processing unit (CPU), and computer are used synonymously to denote a device capable of executing the instructions and communicating with input/output interfaces and/or peripheral devices, and that the terms processor, hardware processor, computer processor, CPU, and computer are intended to encompass single or multiple devices, single core and multicore devices, and variations thereof.

From the description herein, it will be appreciated that the present disclosure encompasses multiple embodiments which include, but are not limited to, the following:

1. A method for performing multi-frequency harmonic acoustography for target identification and border detection, the method comprising: providing a focused confocal transducer having a piezoelectric element and a hydrophone positioned centrally in the piezoelectric element; focusing ultrasonic waves at first and second frequencies from the transducer on a target of interest; wherein the two waves interfere at a focal plane within the target to generate a third acoustic wave and wherein the target absorbs energy and emits its own unique vibration at the difference frequency (Δf) as well as its harmonics; recording the unique vibration with the hydrophone; and determining one or more mechanical properties of the target through detection and analysis of the third acoustic wave using a mathematical model implemented by a processor executing instructions stored in a non-transitory memory accessible by the processor.

2. The system or method of any preceding or subsequent embodiment, wherein said one or more mechanical properties comprise the target's elastic and viscosity properties.

3. The system or method of any preceding or subsequent embodiment, wherein said mathematical model allows for absolute quantitative measurement of tissue properties in terms of properties selected from the group consisting of: elastic modulus, bulk modulus, shear modulus, shear velocity, density, and viscosity.

4. The system or method of any preceding or subsequent embodiment, wherein analysis of the third acoustic wave includes analysis of the harmonics.

5. The system or method of any preceding or subsequent embodiment, wherein the mechanical properties of the target are selected from the group consisting of: convolution of tissue type, size, of the target adjacent tissue, and physiologic or disease state of the target.

6. The system or method of any preceding or subsequent embodiment, wherein determining one or more mechanical properties of the target comprises acquiring a beat frequency generated from the intersection of the first wave and the second wave.

7. The system or method of any preceding or subsequent embodiment, the method further comprising: correlating the acquired beat frequency to the one or more mechanical properties of the tissue.

8. The system or method of any preceding or subsequent embodiment, wherein correlating the acquired beat frequency comprises: generating a database of tissue vibroacoustic responses; and determining the one or more mechanical properties of the target using the database and acquired beat frequency.

9. The system or method of any preceding or subsequent embodiment, the method further comprising: acquiring data relating to the transient response of the unique vibration; and characterizing the one or more mechanical properties of the target as a function of the transient response.

10. The system or method of any preceding or subsequent embodiment, further comprising: measuring the targets viscoelastic response to inertial response after the unique vibration has stopped; and characterizing the one or more mechanical properties of the target as a function of the transient response.

11. A system for performing multi-frequency harmonic acoustography for target identification and border detection, the system comprising: a focused confocal transducer having a piezoelectric element and a hydrophone positioned centrally in the piezoelectric element; a signal processing circuit; the signal processing circuit comprising a processor and a non-transitory memory storing instructions executable by the processor which, when executed, perform steps comprising: causing the transducer to emit ultrasonic waves at first and second frequencies from the transducer on a target of interest; wherein the two waves interfere at a focal plane within the target to generate a third acoustic wave and wherein the target absorbs energy and emits its own unique vibration at the difference frequency (Δf) as well as its harmonics; recording the unique vibration with the hydrophone; and determining one or more mechanical properties of the target through detection and analysis of the third acoustic wave using a mathematical model implemented by the processor in the signal processing circuit executing instructions stored in the memory.

12. The system or method of any preceding or subsequent embodiment, wherein said one or more mechanical properties comprise the target's elastic and viscosity properties.

13. The system or method of any preceding or subsequent embodiment, wherein said mathematical model allows for absolute quantitative measurement of tissue properties in terms of properties selected from the group consisting of: elastic modulus, bulk modulus, shear modulus, shear velocity, density, and viscosity.

14. The system or method of any preceding or subsequent embodiment, wherein analysis of the third acoustic wave includes analysis of the harmonics.

15. The system or method of any preceding or subsequent embodiment, wherein the one or more mechanical properties of the target are selected from the group consisting of: convolution of tissue type, size, of the target adjacent tissue, and physiologic or disease state of the target.

16. The system or method of any preceding or subsequent embodiment, wherein determining one or more mechanical properties of the target comprises acquiring a beat frequency generated from the intersection of the first wave and the second wave.

17. The system or method of any preceding or subsequent embodiment, the steps further comprising: correlating the acquired beat frequency to the mechanical properties of the tissue.

18. The system or method of any preceding or subsequent embodiment, wherein correlating the acquired beat frequency comprises: generating a database of tissue vibroacoustic responses; and determining the mechanical properties of the target using the database and acquired beat frequency.

19. The system or method of any preceding or subsequent embodiment, the steps further comprising: acquiring data relating to the transient response of the unique vibration; and characterizing the mechanical properties of the target as a function of the transient response.

20. The system or method of any preceding or subsequent embodiment, further comprising: measuring the targets viscoelastic response to inertial response after the unique vibration has stopped; and characterizing the mechanical properties of the target as a function of the transient response.

21. The system or method of any preceding or subsequent embodiment, wherein the one or more mechanical properties of the target tissue comprise a boundary between malignant and normal tissue within the target tissue.

22. A method for performing multi-frequency harmonic acoustography for target identification and border detection, the method comprising: providing a focused confocal transducer having a piezoelectric element and a hydrophone positioned centrally in the piezoelectric element; focusing ultrasonic waves at first and second frequencies from the transducer on a target of interest; wherein the two waves interfere at a focal plane within the target to generate a third acoustic wave and wherein the target absorbs energy and emits its own unique vibration at the difference frequency (Δf) as well as its harmonics; recording the unique vibration with the hydrophone; and ascertaining mechanical properties of the target through detection and analysis of the third acoustic wave using a mathematical model implemented by a processor executing instructions stored in a non-transitory memory accessible by the processor.

23. The system or method of any preceding or subsequent embodiment, wherein said mechanical properties comprise the target's elastic and viscosity properties.

24. The system or method of any preceding or subsequent embodiment, wherein said mathematical model allows for absolute quantitative measurement of tissue properties in terms of properties selected from the group consisting of elastic modulus, bulk modulus, shear modulus, shear velocity, density, and viscosity.

25. The system or method of any preceding or subsequent embodiment wherein analysis of the third acoustic wave includes analysis of the harmonics.

26. The system or method of any preceding or subsequent embodiment, wherein the mechanical properties of the target are selected from the group consisting of convolution of tissue type, size, and adjacent tissue and unique to the physiologic or disease state of the tissue of interest.

27. A system for performing multi-frequency harmonic acoustography for target identification and border detection, the system comprising: a focused confocal transducer having a piezoelectric element and a hydrophone positioned centrally in the piezoelectric element; a signal processing circuit; the signal processing circuit comprising a processor and a non-transitory memory storing instructions executable by the processor which, when executed, perform steps comprising: causing the transducer to emit ultrasonic waves at first and second frequencies from the transducer on a target of interest; wherein the two waves interfere at a focal plane within the target to generate a third acoustic wave and wherein the target absorbs energy and emits its own unique vibration at the difference frequency (Δf) as well as its harmonics; recording the unique vibration with the hydrophone; and ascertaining mechanical properties of the target through detection and analysis of the third acoustic wave using a mathematical model implemented by the processor in the signal processing circuit executing instructions stored in the memory.

28. The system or method of any preceding or subsequent embodiment, wherein said mechanical properties comprise the target's elastic and viscosity properties.

29. The system or method of any preceding or subsequent embodiment, wherein said mathematical model allows for absolute quantitative measurement of tissue properties in terms of properties selected from the group consisting of elastic modulus, bulk modulus, shear modulus, shear velocity, density, and viscosity.

30. The system or method of any preceding or subsequent embodiment, wherein analysis of the third acoustic wave includes analysis of the harmonics.

31. The system or method of any preceding or subsequent embodiment, wherein the mechanical properties of the target are selected from the group consisting of convolution of tissue type, size, and adjacent tissue and unique to the physiologic or disease state of the tissue of interest.

As used herein, the singular terms “a,” “an,” and “the” may include plural referents unless the context clearly dictates otherwise. Reference to an object in the singular is not intended to mean “one and only one” unless explicitly so stated, but rather “one or more.”

As used herein, the term “set” refers to a collection of one or more objects. Thus, for example, a set of objects can include a single object or multiple objects.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to +100, such as less than or equal to +5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

TABLE 1
Average power (dBm) and lateral displacements (mm) for Agar two-
layered phantoms.

| | | | Measured | Calculated |
|---|---|---|---|---|
| | Agar Type | Mean | Lateral | Lateral |
| | Concentration | Power | Distance | Distance |
| Line Pair Type | (%) | (dBm) | (mm) | (mm) |
| 15 mm and 15 mm | 2.00 | −4.78 | 15.00 | 15.20 |
| | 4.00 | −7.93 | 15.00 | 15.20 |
| 12 mm and 19 mm | 2.00 | −1.22 | 12.00 | 11.70 |
| | 4.00 | −2.75 | 19.00 | 18.50 |
| 6 mm and 25 mm | 2.00 | 0.02 | 6.00 | 5.40 |
| | 4.00 | −2.19 | 25.00 | 24.20 |
| 4 mm and 27 mm | 2.00 | 0.19 | 4.00 | 3.50 |
| | 4.00 | −1.52 | 27.00 | 27.10 |

TABLE 2
Average power (dBm) and lateral displacements (mm) for Gelatin line-
pair phantoms.

| | | | Measured | Calculated |
|---|---|---|---|---|
| | Gelatin Type | Mean | Lateral | Lateral |
| | Concentration | Power | Distance | Distance |
| Line Pair Type | (%) | (dBm) | (mm) | (mm) |
| 17 mm and 15 mm | 10.00 | −10.80 | 17.00 | 16.50 |
| | 20.00 | −17.58 | 15.00 | 15.00 |
| 12 mm and 20 mm | 10.00 | −7.12 | 12.00 | 11.00 |
| | 20.00 | −20.01 | 20.00 | 21.00 |
| 7 mm and 24 mm | 10.00 | −3.14 | 7.00 | 7.80 |
| | 20.00 | −6.42 | 24.00 | 22.20 |
| 6 mm and 25 mm | 10.00 | −11.66 | 6.00 | 5.00 |
| | 20.00 | −12.26 | 25.00 | 25.00 |

TABLE 3
Three-layered and four-layered phantom results for gelatin and agar.

| Phantom | | | | | | | |
|---|---|---|---|---|---|---|---|
| Type | Three-Layered | | | | | Four-Layered | |
| Concen- | 15% | 2% | 10% | 20% | 3% | 20% | 3% |
| tration | Gelatin | Agar | Gelatin | Gelatin | Agar | Gelatin | Agar |
| Mean Power | −6.17 | −4.75 | −4.77 | −13.51 | −15.03 | −11.96 | −14.67 |
| (dBm) | | | | | | | |
| Measured | 10.00 | 14.00 | 10.00 | 5.00 | 4.00 | 10.00 | 4.00 |
| Lateral | | | | | | | |
| Distance | | | | | | | |
| (mm) | | | | | | | |
| Calculated | 9.80 | 13.60 | 10.30 | 4.20 | 3.30 | 10.70 | 3.80 |
| Lateral | | | | | | | |
| Distance | | | | | | | |
| (mm) | | | | | | | |

TABLE 4
Mean elastic modulus, viscosity, and time constant data for PVA, gelatin, and agar.

| | | | | | Long term | | | | |
|---|---|---|---|---|---|---|---|---|---|
| | Elastic Modulus | | | | Shear Modulus | | | | |
| | (kPa) | | | | (MPa sec) | | Mean | | Time |
| Phantom | 600 | 800 | Mean | Standard | 600 | 800 | (MPa | Standard | Constant |
| Type | μm | μm | (kPa) | Deviation | μm | μm | sec) | Deviation | (sec) |
| 14% PVA | 5.722 | 5.598 | 5.66 | 0.158 | 1.105 | 0.883 | 0.994 | 0.04 | 175.657 |
| 17% PVA | 9.552 | 9.319 | 9.435 | 0.2 | 1.785 | 1.467 | 1.626 | 0.075 | 172.33 |
| 20% PVA | 33.563 | 33.866 | 33.715 | 1.015 | 4.693 | 3.824 | 4.259 | 0.273 | 126.317 |
| 10% | 15.823 | 16.873 | 16.348 | 0.897 | 2.83 | 2.71 | 2.77 | 0.128 | 169.424 |
| Gelatin | | | | | | | | | |
| 15% | 43.518 | 46.903 | 45.21 | 2.243 | 6.39 | 6.358 | 6.374 | 0.309 | 140.988 |
| Gelatin | | | | | | | | | |
| 20% | 62.01 | 69.907 | 65.959 | 3.087 | 8.649 | 8.957 | 8.803 | 0.445 | 133.458 |
| Gelatin | | | | | | | | | |
| 2% Agar | 106.497 | 102.778 | 104.638 | 3.911 | 5.595 | 6 | 5.797 | 0.21 | 55.405 |
| 2.5% | 137.034 | 133.918 | 135.476 | 4.647 | 9.122 | 10.045 | 9.583 | 0.607 | 70.737 |
| Agar | | | | | | | | | |
| 3% Agar | 199.049 | 191.284 | 195.166 | 4.281 | 13.575 | 14.913 | 14.244 | 0.84 | 72.984 |

TABLE 5
Ex vivo porcine liver, porcine gallbladder, and rat liver
elastic modulus, viscosity, and time constant

| | | | | | Long | | | |
|---|---|---|---|---|---|---|---|---|
| | | | | | term | | | |
| | | | | | Shear | | | |
| | | Elastic | | | Modulus | Mean | | Time |
| Tissue | | Modulus | Mean | Standard | (MPa | (MPa | Standard | Constant |
| Type | Depth | (kPa) | (kPa) | Deviation | sec) | sec) | Deviation | (sec) |
| Porcine | 300 | 2.575 | 2.553 | 0.085 | 0.138 | 0.135 | 0.006 | 52.879 |
| Liver | μm | | | | | | | |
| | 400 | 2.531 | | | 0.132 | | | |
| | μm | | | | | | | |
| Rat Liver | 200 | 2.89 | 2.758 | 0.094 | 0.154 | 0.147 | 0.007 | 53.299 |
| | μm | | | | | | | |
| | 300 | 2.626 | | | 0.14 | | | |
| | μm | | | | | | | |
| Porcine | 100 | 4.73 | 4.73 | 0.735 | 0.176 | 0.176 | 0.033 | 37.209 |
| Gallbladder | μm | | | | | | | |

What is claimed is:

1. A method for performing multi-frequency harmonic acoustography for target identification and border detection, the method comprising:
providing a focused confocal transducer having a piezoelectric element and a hydrophone positioned centrally in the piezoelectric element;
focusing ultrasonic waves at first and second frequencies from the transducer on a target of interest;
wherein the two waves interfere at a focal plane within the target to generate a third acoustic wave and wherein the target absorbs energy and emits its own unique vibration at the difference frequency (Δf) as well as its harmonics;
recording the unique vibration with the hydrophone; and
determining one or more mechanical properties of the target through detection and analysis of the third acoustic wave using a mathematical model implemented by a processor executing instructions stored in a non-transitory memory accessible by the processor.

2. The method of claim 1, wherein said one or more mechanical properties comprise the target's elastic and viscosity properties.

3. The method of claim 1, wherein said mathematical model allows for absolute quantitative measurement of tissue properties in terms of properties selected from the group consisting of: elastic modulus, bulk modulus, shear modulus, shear velocity, density, and viscosity.

4. The method of claim 1, wherein analysis of the third acoustic wave includes analysis of the harmonics.

5. The method of claim 1, wherein the mechanical properties of the target are selected from the group consisting of: convolution of tissue type, size, of the target adjacent tissue, and physiologic or disease state of the target.

6. The method of claim 1, wherein determining one or more mechanical properties of the target comprises acquiring a beat frequency generated from the intersection of the first wave and the second wave.

7. The method of claim 6, the method further comprising:
correlating the acquired beat frequency to the one or more mechanical properties of the tissue.

8. The method of claim 7, wherein correlating the acquired beat frequency comprises:
generating a database of tissue vibroacoustic responses; and
determining the one or more mechanical properties of the target using the database and acquired beat frequency.

9. The method of claim 1, the method further comprising:
acquiring data relating to the transient response of the unique vibration; and
characterizing the one or more mechanical properties of the target as a function of the transient response.

10. The method of claim 9, further comprising:
measuring the targets viscoelastic response to inertial response after the unique vibration has stopped; and
characterizing the one or more mechanical properties of the target as a function of the transient response.

11. A system for performing multi-frequency harmonic acoustography for target identification and border detection, the system comprising:
a focused confocal transducer having a piezoelectric element and a hydrophone positioned centrally in the piezoelectric element;
a signal processing circuit;
the signal processing circuit comprising a processor and a non-transitory memory storing instructions executable by the processor which, when executed, perform steps comprising:
causing the transducer to emit ultrasonic waves at first and second frequencies from the transducer on a target of interest;
wherein the two waves interfere at a focal plane within the target to generate a third acoustic wave and wherein the target absorbs energy and emits its own unique vibration at the difference frequency (Δf) as well as its harmonics;
recording the unique vibration with the hydrophone; and
determining one or more mechanical properties of the target through detection and analysis of the third acoustic wave using a mathematical model implemented by the processor in the signal processing circuit executing instructions stored in the memory.

12. The system of claim 11, wherein said one or more mechanical properties comprise the target's elastic and viscosity properties.

13. The system of claim 11, wherein said mathematical model allows for absolute quantitative measurement of tissue properties in terms of properties selected from the group consisting of: elastic modulus, bulk modulus, shear modulus, shear velocity, density, and viscosity.

14. The system of claim 11, wherein analysis of the third acoustic wave includes analysis of the harmonics.

15. The system of claim 11, wherein the one or more mechanical properties of the target are selected from the group consisting of: convolution of tissue type, size, of the target adjacent tissue, and physiologic or disease state of the target.

16. The system of claim 11, wherein determining one or more mechanical properties of the target comprises acquiring a beat frequency generated from the intersection of the first wave and the second wave.

17. The system of claim 16, the steps further comprising:
correlating the acquired beat frequency to the mechanical properties of the tissue.

18. The system of claim 17, wherein correlating the acquired beat frequency comprises:
generating a database of tissue vibroacoustic responses; and
determining one or more mechanical properties of the target using the database and acquired beat frequency.

19. The system of claim 11, the steps further comprising:
acquiring data relating to the transient response of the unique vibration; and
characterizing the mechanical properties of the target as a function of the transient response.

20. The system of claim 19, further comprising:
measuring the targets viscoelastic response to inertial response after the unique vibration has stopped; and
characterizing the mechanical properties of the target as a function of the transient response.

21. The system of claim 11, wherein the one or more mechanical properties of the target tissue comprise a boundary between malignant and normal tissue within the target tissue.
